(19) United States
(12) Patent Application Publication
(10) Pub. No.: US 2006/0003385 A1
(43) Pub. Date: Jan. 5, 2006

(54) Mass intensity profiling system and uses thereof

(75) Inventors: Frank Reinaldo Morales Aguilera, Saint-Jean Chrysostome (CA); Denis Faubert, Montreal (CA); Marguerite Boulos, Laval (CA); John Shing-Chun Tsang, Somerville, MA (US); Michael Hu, Boston, MA (US); Joachim Bernhard Ostermann, Toronto (CA); Paul Edward Kearney, Montreal (CA); Pierre Thibault, Ile Bizard (CA)

Correspondence Address: CLARK & ELBING LLP, 101 FEDERAL STREET, BOSTON, MA, 02110, US

(73) Assignee: Caprion Pharmaceuticals, Montreal (CA)

(21) Appl. No.: 11/188,954

(22) Filed: Jul. 25, 2005

Related U.S. Application Data
(62) Division of application No. 10/293,076, filed on Nov. 13, 2002.
(60) Provisional application No. 60/338,578, filed on Nov. 13, 2001.

(51) Int. Cl. C12Q 1/68; G01N 33/53; G06F 19/00

(52) U.S. Cl. 435/7.1; 702/19; 436/86

(57) ABSTRACT

The present invention is directed to computer automated methods and systems for identifying and characterizing biomolecules in a biological sample. Mass spectrometry measurements are obtained on biomolecules in a sample. These measurements are analyzed to determine the abundance of the biomolecules in the sample, and the abundance measurements are coupled with one or more distinguishing characteristics of biomolecules they are associated with, thereby permitting computer-mediated comparison of abundances of biomolecules from multiple biological samples.

### CROSS REFERENCE TO RELATED APPLICATIONS

This application claims benefit of priority from U.S. Provisional Application No. 60/338,578, filed Nov. 13, 2001, hereby incorporated by reference.

### BACKGROUND OF THE INVENTION

The invention relates to the fields of mass spectrometry, bioinformatics, and computational molecular biology. In particular, this invention relates to the automation of biomolecule quantification.

Genomic and proteomic research efforts in recent years have vastly improved our understanding of the molecular basis of life at a global cellular and tissue scale. In particular, it is increasingly clear that the temporal and spatial expression of an organism's biomolecules is responsible for life's processes—processes occurring in both health and in sickness. Science has progressed from understanding how genetic defects cause hereditary disorders, to an understanding of the importance of the interaction of multiple genetic defects together with environmental factors in the etiology of complex medical disorders, such as cancer. In the case of cancer, scientific evidence demonstrates the key causative roles of altered expression of, and multiple defects in, several pivotal genes and their protein products. Other complex diseases have similar molecular underpinnings. Accordingly, the more complete and reliable a correlation that can be established between expression of an organism's biomolecules and healthy or diseased states, the better diseases can be diagnosed and treated. Methods that permit efficient and rapid quantification of biomolecule expression from biological samples that may contain tens of thousands of different biomolecules of a particular type (e.g., protein, lipids, nucleic acids, carbohydrates, metabolites, and combinations thereof) are necessary to provide the best possible chance to determine such correlations. For example, proteomic data reflects the true expression levels of functional molecules and their post-translational modifications, which cannot be accurately predicted from other data types such as gene expression profiling.

A central goal of proteomics, which involves the systematic identification and characterization of proteins in a sample, is to be able to compare the protein composition between two or more samples. Critical to achieving this goal is the ability to identify all the proteins that are present in only one sample or type of sample and any proteins that are present in several samples or types of samples but differ in abundance. The complexity and dynamic nature of the proteomes of living beings, however, as well as limitations in sample quantity and stability, provide enormous challenges in identifying the amino acid sequence and the source protein(s) of the peptidic material present in a sample, in quantifying the relative abundance of the different peptides or source proteins present in the sample, and in providing complete enough data about a sample to produce an accurate snapshot of the proteome. The complexity of the proteomes and of these tasks further makes performing such proteome-wide analyses and comparisons difficult to accomplish in a reasonable time frame.

Comparability itself is also an issue. The methods by which two biomolecules are judged to be the same or comparable depend on the methods employed to identify a particular biomolecule within a field of biomolecules and the completeness of the data gathered from a sample. Usually, a comparison of all the proteins in a sample is accomplished by two-dimensional (2D) gel electrophoresis, which resolves a complex protein mixture into hundreds or thousands of spots, which have characteristic migratory positions for particular proteins. Gel patterns can be directly comparable with correction of migration variables if the gel and sample were properly prepared and run, but gel reproducibility is quite variable from lab to lab or even with different lots of ampholines. Each spot, in theory, represents one protein, and the intensity of each spot is taken as a measure for the amount of the protein present. The protein that is present in this spot can then be more fully identified by mass spectrometry or other methods; however, the further identification of a single protein spot, let alone the whole field of spots, can involve considerable time, effort, and expense. The 2D electrophoresis approach also has several other drawbacks, the most important of which is the difficulty of identifying membrane proteins. In general, 2-D electrophoresis has problems with the exclusion of highly hydrophobic molecules, and with the detection of highly charged (very acidic or very basic) molecules, as well as of very small or very large molecules. In addition, the detection of low or even moderate abundance proteins is difficult and may require that several gels be run to collect enough material for sequence analysis. 2D gel spots can also be quite large, which dilutes the protein over a large part of the gel, rendering detection and accurate quantification of proteins more difficult. Additionally, co-migration of proteins, particularly of closely related or variant proteins, can interfere with both proper identification and quantification of the specific proteins.

One-dimensional (1D) gel electrophoresis, on the other hand, is a generally applicable tool to separate proteins that at least allows the study of both soluble and membrane proteins. However, when complex mixtures of proteins are analyzed, only 50 to 100 protein bands are typically detectably produced in the separation, and a single band in a 1D gel may, therefore, contain more than a single protein. For this reason, the intensity of one band does not typically reflect the abundance of a single protein in the sample, and identification likewise becomes more problematic. Mass spectrometry, for example, of a single band will lead to the identification of not just one but several (e.g. 10 to 20) proteins that are present in the band at different concentrations.

Mass spectrometry itself is another method of choice for analyzing complex mixtures of molecules, such as the contents of cells, or cellular components. When combined with appropriate methods of chromatography to allow separation and purification of biomolecules, mass spectrometry provides a start point for producing and analyzing data for the identification and quantification of biomolecules, and for patterns that liken or distinguish different samples. At its most basic, mass spectrometry produces data about the mass of biomolecules, and their intensity (ion counts) for a particular scan. Fragmentation patterns for specific molecules can also be produced, but these characteristic spectra, which can be used to further identify the molecule, are unlinked to the quantitative data (ion counts) produced in the initial scan. Secondary efforts are required to derive structural information from this basic data, or, in the case of polymers such as DNA or proteins, to obtain sequence information from the fragmentation patterns, to determine the source protein from the sequence information, and to couple sequence/identity information to quantification data.

One quantitative mass spectrometric technique relies on coupling different isotopic tags to the peptides of each sample to be analyzed. An example of this methodology is referred to as isotope-coded affinity tag (ICAT) (see Han et al. (2001) Nat. Biotechnol. 19: 946-51 (PMID: 11581660)). This method consists of derivatizing proteins, such as with alkylating agents containing a reactive group specific to cysteine residues, a linker chain, and a biotinylated moiety. The alkylating agent includes a light and a heavy version corresponding to 8 hydrogen atoms (light) or 8 deuterium atoms (heavy) in the linker chain. When comparing two samples, all the peptides from one sample are tagged with the light tag, and all the peptides from the other sample are tagged with the heavy tag. Both samples are then mixed, digested with trypsin, and analyzed simultaneously. In the mass spectrum, ions pairs that correspond to the same peptide but differ by the exact mass difference (8 Da) between the heavy and light tag are then identified. These ions then correspond to the same peptide but are derived from the two different samples. This method allows for the direct comparison of the abundance of corresponding peptides from the two samples. Despite permitting direct comparison of samples, this technique generally has the limitation that all peptides containing cysteine residues must be chemically modified before they are analyzed. Such modifications come at an additional expense in both money and time. They can also have a cost in accuracy if the reaction does not go to completion, or the delays due to processing time lead to protein degradation. Furthermore, the chemical modification requires the presence of a specific amino acid, cysteine, in the peptide, which means that the majority of peptides are not suitable for the analysis. This requirement greatly reduces the applicability of this approach to a wide range of proteins. The ICAT approach can also generate interfering intensities from biotinylated fragment ions in MS/MS experiments, hampering the ability to determine peptide sequence information.

Another labeling method uses light and heavy isotopes of water. Tryptic peptides from different protein pools are labeled at the C-terminus with ¹⁶O and ¹⁸O water. This method has been used to distinguish between b- and y-type fragment ions in MS/MS experiments (see Schevshenko et al. (1997) Rapid Commun. Mass Spectrom. 11: 1015-1024). The method has also been used for monitoring the differential expression of proteins in two serotypes of adenovirus (see Yao et al. (2001) Anal. Chem. 73: 2836-2842). As above, protein pools are digested separately, labeled, and combined for analysis by mass spectrometry. Expression profiles are then obtained based on the ratio of heavy to light ions. This method also requires that the peptides or proteins be labeled before analysis, and thus, like ICAT may suffer from incomplete reactions, substrate insusceptibility, extra cost, and extra preparation time made all the more costly by the possible detriment to limited and potentially unstable samples. These issues are exacerbated by the additional challenges of preparing such samples from living organisms.

Existing methods for biomolecule identification or characterization are therefore in need of improvement in their ability to perform rapid, accurate, automated, and economical as well as qualitative, quantitative, and specific determinations of the components of a biological sample. For example, there exists a need for methods using mass spectrometry to determine the abundance of peptides in a sample that does not require chemical modification of the peptides. Furthermore, there is a continuing and significant need to be able to readily compare the relative abundances of proteins between biological samples, and to identify and characterize proteins as targets for drug discovery. The present invention fulfills these needs and further provides other related advantages.

### SUMMARY OF THE INVENTION

The present invention features computer automated methods and systems for identifying and characterizing biomolecules in a biological sample. In these methods, mass spectrometry measurements are obtained on biomolecules in a sample. These measurements are then analyzed by the methods described herein to determine the abundance of the biomolecules in the sample, and the abundance measurements are coupled with one or more distinguishing characteristics of biomolecules they are associated with, thereby permitting computer-mediated comparison of abundances of biomolecules from multiple biological samples. We refer to this technology as “MIPS” or mass intensity profiling system. This automated-MIPS technology for screening biological samples and comparing their mass intensity profiles permits rapid and efficient identification of individual biomolecules whose presence, absence, or altered expression is associated with a disease or a condition of interest. Such biomolecules (for example, proteins) are potentially useful as therapeutic agents, as targets for therapeutic intervention, or as markers for diagnosis, prognosis, and evaluating response to treatment. MIPS technology also permits rapid identification of sets of biomolecules whose pattern of expression is associated with a disease or condition of interest; such sets of biomolecules provide a collection of biological markers for potential use in diagnosis, prognosis, and evaluating response to treatment.

In one aspect, the invention features a method for determining an abundance of a biomolecule in a biological sample. In general, the method includes the steps of providing a biological sample containing a plurality of biomolecules; generating a plurality of ions of the biomolecules; performing mass spectrometry measurements on the plurality of ions, thereby obtaining ion counts for the biomolecules; assigning an ion to a biomolecule; and integrating the ion counts of the biomolecule, thereby determining the abundance of the biomolecule in the biological sample.

In particular, the invention features methods and systems for the determination of the abundance of peptides in a sample, but the following methods may be applied to other biomolecules as well. The invention further features methods and systems that can be used to compare expression levels of peptides between two or more samples, as well as methods to compare expression levels of proteins between two or more samples. These methods are based on the analysis of data from mass spectrometry.

In various embodiments, MIPS can be used to determine the abundance of peptidic material present in one or more LC/MS scans. MIPS may be used to determine the abundance for all the precursors present, all precursors which have been matched with spectra generated by MS/MS, or precursors as limited by a list (inclusion or exclusion), a query, or set of queries.

In one embodiment, MIPS can be used to calculate the abundance of one or more pepti des without a determination of amino acid sequence or full-length protein identification. Alternatively, MIPS can be used to calculate the abundance of one or more peptides with a determination of amino acid sequence but without full-length protein identification. In another embodiment, MIPS can be used to calculate the abundance of one or more peptides and perform peptide and full-length protein identification without prior amino acid sequence determination, through methods including, but not limited to, peptide mass fingerprinting (see, for example, Cottrell (1994) Pept. Res. 7: 115-24). In still another embodiment, MIPS can be used to calculate the abundance of one or more peptides, to determine the amino acid sequence and to identify the full-length protein of which a peptide is a constituent. The above methods may also be modified to include determining an amino acid sequence but not relying upon this information in determining the identity of a full-length protein. The abundance of a full-length protein so identified can be calculated, based on the abundance of one or more of its constituent peptides. These methods can also be used to match or compare the abundance of the same peptide or protein between two or more samples or the abundance of different peptides or proteins in the same or a different sample. Comparisons between samples may include comparing data from a patient to a reference, manipulated, representative, combined, or theoretical samples.

In various embodiments, MIPS can be used to identify one or more constituent peptides of an identified full-length protein, and to calculate the abundance of one or more of the identified constituent peptides. Such abundance data may be used in turn to calculate or recalculate the abundance of a full-length protein.

In another embodiment, MIPS can be used to query the abundance of one or more peptides or full-length proteins in one or more samples, with or without prior calculation of said abundances, and with or without prior identification of the one or more peptides or proteins. Queries may be manually entered or part of an automated process, such as a list of ions to exclude from MS/MS. Queries may take place prior to data acquisition and wholly or in part determine the data collected, or they may occur post-data acquisition to mine data.

In various embodiments, the calculation of full-length protein abundance may be based upon the abundance of one or more constituent peptides. Such peptides may have been previously identified, or they may be sought (automatically or manually) and their abundances calculated based on the predicted peptides for the particular full-length protein.

In various embodiments, the calculation of peptide abundance may be absolute or relative. In general, abundance is determined by a sum of ion counts based on a consistent choice within a sample, for example, a subset of charge states, isotopes, modified states, or a combination thereof.

In various embodiments, comparisons may be to the same or a different peptide or protein. Comparisons may also be to homologous or analogous peptides or proteins, individual peptides or proteins, or peptides or proteins representative of a family or group. Comparisons may also be to unrelated peptides or proteins. Data may be extrapolated and statistically analyzed for individual peptides and/or proteins, and/or groupings thereof. Data used for comparison may be from within the same set of sample data, and/or from one or more other sets of data including, but not limited to, reference, manipulated, representative, combined, and/or theoretical samples.

In various embodiments, matching a peptide or peptides, protein or proteins can be based on experimental or theoretical retention time. Theoretical retention time may be calculated to take into account error, charge state, theoretical and/or adjusted charge state, or isotopes, and they may also be adjusted using one or more internal standards, including-those exogenous to a sample. Other methods include, but are not limited to, pattern monitoring, such as isotope pattern monitoring (peptide recognition is based on the isotopic model of a given peptide (height, width and distance of peaks)). Such matching may be for purposes including, but not limited to, querying data, comparisons, or uniting data from separate data sources including, but not limited to, separate scans, spectra, sequence information, annotations, and combinations thereof.

In various embodiments, a peptide or protein in a sample for which an abundance can be calculated may be used to generate a list of one or more peptides or proteins, which may in turn be combined with other lists or used directly or indirectly for querying, matching, or governing data gathering, such as selection for spectra determination in further analysis of the same or another sample.

The invention further features a computer implemented method for determining abundance of a biomolecule in a biological sample. The computer implemented method generally includes the steps of inputting mass spectrometry data comprising ion counts for a plurality of biomolecules; assigning an ion to a biomolecule; and integrating the ion counts of the biomolecule, thereby determining the abundance of the biomolecule in the biological sample.

In another aspect, the invention features a computer-readable memory that includes a program for determining abundance of a biomolecule in a biological sample including computer code that receives as input mass spectrometry data comprising ion counts for a plurality of biomolecules; computer code that assigns an ion to a biomolecule; and computer code that integrates the ion counts of the biomolecule, thereby determining the abundance of the biomolecule in the biological sample.

In yet another aspect, the invention features a system for determining abundance of a biomolecule in a biological sample including a mass spectrometry data input module that receives data including ion counts for a plurality of biomolecules; an ion assigning module responsive to the data input module, wherein the ion assigning module assigns an ion to a biomolecule; and an ion integrating module responsive to the ion assigning module, wherein the ion integrating module integrates ion counts of the biomolecule, thereby determining the abundance of the biomolecule in the biological sample. In one embodiment, the system includes a processor and a memory coupled to the processor, wherein the memory encodes the data input module, the ion assigning module, and the ion integrating module.

In another aspect, the invention features a method for displaying information on abundance of a biomolecule in a biological sample to a user including the steps of inputting mass spectrometry data comprising ion counts for a plurality of biomolecules; assigning an ion to a biomolecule; integrating the ion counts of the biomolecule, thereby determining the abundance of the biomolecule in the biological sample; and displaying the abundance of the biomolecule. In one embodiment, the method further includes storing the abundance of the biomolecule in a memory.

In various embodiments of any of the aforementioned aspects, the biomolecule is underivatized or unlabeled. The biomolecule may also be cleaved biomolecule. In preferred embodiments, the biomolecule is cleaved with an enzyme. In general, however, the methods do not require modification other than cleavage, such as isotope-labeling or akylation, of the biomolecules, i.e., cleaved biomolecules may be underivatized or unlabeled. In yet other preferred embodiments, the invention further features assaying two or more biological samples. The invention, if desired, features the inclusion of one or more internal standards in the biological sample. In still another embodiment, a computer procedure assigns the ion to the biomolecule by calculating an uncharged mass for the ion. Alternatively, ions may be assigned to biomolecules through mass fingerprinting, e.g., peptide mass fingerprinting. In yet another embodiment, a computer procedure integrates ion counts of the ions corresponding to the biomolecule. Preferably, the integration is over one or more charge states, isotopes, scans, fragments of the biomolecule, fractions of a separation, or a combination thereof. In other embodiments, the invention further features separating the plurality of biomolecules prior to MS analysis. Typically, such separation is carried out using standard methods known in the art. These methods include, without limitation, chromatography, electrophoresis, immunoisolation (e.g., using magnetic beads), or centrifugation. The retention time of an ion may be corrected using one or more internal standards.

In various other embodiments of any of the aforementioned aspects, the biomolecule is typically a protein or modified protein. Preferably, the protein is obtained from an isolated organelle. Exemplary isolated organelles include, without limitation, mitochondria, chloroplasts, ER, Golgi, endosomes, lysosomes, phagosomes, peroxisomes, secretory vesicles, transport vesicles, nuclei, and plasma membrane. Proteins obtained from other cellular components are also useful in the invention. These proteins include cytosolic or cytoskeletal proteins.

In preferred embodiments, mass spectrometry measurements are obtained to gather structural or sequence information of an ion of the biomolecule, e.g., through MS/MS analysis. Biomolecules or ions thereof may be selected for structural or sequence analysis (e.g., MS/MS analysis) by a query. In one embodiment, an inclusion or exclusion list is used to determine which ions will be subjected to structural or sequence analysis. The methods and systems of the invention further feature the use of a computer procedure to identify a protein comprising the sequence of the ion from a database. Exemplary procedures include Mascot®, Protein Lynx Global Server, SEQUEST®/TurboSEQUEST, PEPSEQ, SpectrumMill, or Sonar MS/MS. Exemplary databases that are searched using such procedures include the Genbank®, EMBL, NCBI, MSDB, SWISS-PROT®, TrEMBL, dbEST, or Human Genome Sequence database. Moreover, the methods and systems include a computer procedure that assigns the ion to the protein identified from a database.

In various other embodiments of any of the aforementioned aspects, the invention features calculating an abundance of the biomolecule relative to a control biological sample and calculating abundances of a plurality of the biomolecules relative to a control biological sample. Typically, abundance measurements of a set of biomolecules are used to diagnose a disease or condition. Additionally, abundance is used to determine a biomolecule to target with a drug. Such targets are identified by evaluating an increase or decrease in abundance or the presence or absence of a biomolecule in the biological sample relative to a control sample. Abundance of a biomolecule may also be used to determine an amount of an isoform of a biomolecule, or of a naturally occurring modification of a biomolecule.

By “biomolecule” is meant any organic molecule that is present in a biological sample, including peptides, polypeptides, proteins, post-translationally modified peptides or proteins (e.g., glycosylated, phosphorylated, or acylated peptides), oligosaccharides, polysaccharides, lipids, nucleic acids, and metabolites.

By “biological sample” (or “sample”) is meant any solid or fluid sample obtained from, excreted by, or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). A biological sample may be a biological fluid obtained from any location (such as blood, plasma, serum, urine, bile, cerebrospinal fluid, aqueous or vitreous humor, or any bodily secretion), an exudate (such as fluid obtained from an abscess or any other site of infection or inflammation), or fluid obtained from a joint (such as a normal joint or a joint affected by disease such as rheumatoid arthritis). Alternatively, a biological sample can be obtained from any organ or tissue (including a biopsy or autopsy specimen) or may comprise cells (whether primary cells or cultured cells) or medium conditioned by any cell, tissue or organ. If desired, the biological sample is subjected to preliminary processing, including preliminary separation techniques. For example, cells or tissues can be extracted and subjected to subcellular fractionation for separate analysis of biomolecules in distinct subcellular fractions, e.g., proteins or drugs found in different parts of the cell. A sample may be analyzed as subsets of the sample, e.g., bands from a gel.

By “assigning an ion to a biomolecule” is meant specifying a biomolecule from which an ion observed in a mass spectrum was generated. The ion may be assigned to a biomolecule or a fragment thereof. Such assignments may be based, for example, on the molecular mass, or other physicochemical characteristic. The assignment can also be made on the basis of determining the molecular mass of the ion and matching that mass with a known biomolecule or on the basis of data, e.g., from MS/MS, that identifies structural or sequence information about the ion, which may be used to search a database.

By “uncharged mass” is meant the mass of the neutral charge state of the biomolecule or a fragment thereof from which an ion is generated.

By “integrating the ion counts of a biomolecule” is meant summing ion counts for data within a defined range of m/z values. The phrase also refers to summing integrated ion counts of two or more ions. For example, ions that are found in different charge states, isotopes, fractions of a separation, scans, or fragments of a biomolecule may be integrated.

By the term “protein” is meant any polymer of two or more individual amino acids linked via a peptide bond that forms when the carboxylcarbon atom of the carboxylic acid group bonded to the alpha-carbon of one amino acid (or amino acid residue) becomes covalently bound to the amino nitrogen atom of amino group bonded to the alpha-carbon of an adjacent amino acid. The term “protein” is understood to include the terms “polypeptide” and “peptide” (which, at times, may be used interchangeably herein) within its meaning, as well as post-translational modifications and fragments thereof. In addition, proteins comprising multiple polypeptide subunits (e.g., insulin receptor, cytochrome b/cl complex, and ribosomes) or other components (for example, an RNA molecule) will also be understood to be included within the meaning of “protein” as used herein. Similarly, fragments of proteins and polypeptides are also within the scope of the invention and may be referred to herein as “proteins,” “polypeptides,” or “peptides,” “tryptic peptides”, or “cleavage fragments.” “Constituent peptides” are peptides whose sequence is a linear subset of the sequence of a larger peptide or full-length protein. As a group, the “constituent peptides” for a particular protein would be a set or subset of those that make up the protein. Usually, this is a subset limited to particular cleavage fragments, such as the set of tryptic peptides that make up a protein. A “full-length protein” refers to a protein encoded by and translated from a messenger RNA (mRNA), and post-translational modifications thereof. Full-length proteins may be identified through database searching via computer procedures as described herein.

By “precursor” is meant a biomolecule, e.g., a potential peptide or protein or one of unknown sequence or identity. Generally it refers to potential peptides in mass spectrometry survey scan data prior to secondary identification efforts, such as sequencing by MS/MS. “Precursors” are frequently identified by comparing their masses or their retention times. Such retention times may be experimental or theoretical. Theoretical retention times are frequently corrected, where one or more internal standards are used to make retention times comparable between samples. Predicted retention times may be used to seek precursors within a scan. “Precursor” is frequent-ly-used interchangeably with “peptide,” and it may be used to distinguish individual constituent peptides from full-length proteins.

By “scan” is meant a mass spectrum from a single sample. Each fraction of a separation that is measured results in a scan. If a biomolecule is located in more than one fraction analyzed, then the mass spectrum for the biomolecule is present in more than one scan.

By “fraction” is meant a portion of a separation. A fraction may correspond to a volume of liquid obtained during a defined time interval, for example, as in LC (liquid chromatography). A fraction may also correspond to a spatial location in a separation such as a band in a separation of a biomolecule facilitated by gel electrophoresis.

By “query” is meant a selection of a particular action. In one example of a query, ions may be subjected to MS/MS based on a list that is stored with the software. Alternatively, one can manually select ions to be subjected to MS/MS. This manual selection is also a query.

By an “underivatized” biomolecule or fragment thereof is meant a biomolecule or fragment thereof that has not been chemically altered from its natural state. Derivitization may occur during non-natural synthesis or during later handling or processing of a biomolecule or fragment thereof.

By an “unlabeled” biomolecule or fragment thereof is meant a biomolecule or fragment thereof that has not been derivatized with an exogenous label (e.g., an isotopic label or radiolabel) that causes the biomolecule or fragment thereof to have different physicochemical properties to naturally synthesized biomolecules.

The methods and systems of the invention provide a number of significant advantages. For example, the methods and systems combine mass spectrometry and data analysis in a way that allows both the identification of proteins and a measure of their abundance. The methods thus allow a correlation of ion intensities of a biomolecule between sample sets in order to compare relative abundance of the original biomolecule from which the biomolecules are derived. Further, the methods are fully automated allowing a comprehensive comparison of an unlimited number of proteins from different data sets. This automation thus allows for protein abundances in two or more samples to be compared. The information from the entire mass spectrum can also be used to determine expression levels. Typically, a large amount of information that is present in the mass spectra is discarded, and only a subset, such as intensities of specific ions, or the sequence of specific peptides, or a list of peptide masses is analyzed. Finally, the use of automation greatly reduces the time necessary for analysis.

Other features and advantages of the invention will be apparent from the following drawings and detailed description, and from the claims.

### BRIEF DESCRIPTION OF THE DRAWINGS

FIG. 1 illustrates an exemplary embodiment of a computer system of this invention.

FIG. 2 shows a flowchart of a method of determining an abundance of a biomolecule in a sample.

FIG. 3 shows a flow chart for a Peptide Abundance Module. Solid rectangles represent processing components of MIPS, dashed rectangles represent processing components that are not within MIPS, and entries without a rectangle are data files.

FIG. 4A shows a mass spectrum scan corresponding to a first elution time in a scan window.

FIG. 4B shows a mass spectrum scan corresponding to a second elution time in a scan window.

FIG. 4C shows a mass spectrum scan corresponding to a third elution time in a scan window.

FIG. 4D shows a mass spectrum scan corresponding to a fourth elution time in a scan window.

FIG. 5 show a mass spectrum illustrating the presence of two charge states.

FIG. 6 shows a mass spectrum illustrating the separation between isotopes of a +3 charge state.

FIG. 7 shows a flow chart for a Peptide Hunter Module.

FIGS. 8A-8I show results for three different m/z values from the HSP90 protein. FIGS. 8A-8C show the reconstructed ion chromatogram, survey scan, and MS/MS of the 924.4 m/z ion. FIGS. 8D-8F show the reconstructed ion chromatogram, survey scan, and MS/MS of the 757.4 m/z ion. FIGS. 8G-8I show the reconstructed ion chromatogram, survey scan, and MS/MS of the 621.8.

FIG. 9 shows a table of the abundances of here m/z values of the HSP90 protein in five samples. The abundances were summed across several 1-D gel bands.

FIGS. 10A-10I show results for three different m/z values from the mutant desmin protein. FIGS. 10A-10C show the reconstructed ion chromatogram, survey scan, and MS/MS of the 561.3 m/z ion. FIGS. 10D-10F show the reconstructed ion chromatogram, survey scan, and MS/MS of the 558.3 m/z ion. FIGS. 10G-10I show the reconstructed ion chromatogram, survey scan, and MS/MS of the 466.7.

FIG. 11 shows a table of the abundances of here m/z values of the mutant desmin protein in five samples. The abundances were summed across several 1-D gel bands.

FIGS. 12A and 12B show differential expression of HSP90 and mutant desmin in U937 cells using MIPS

FIG. 13 shows a flow chart for a Peptide Abundance Module processing matched samples (N/T), such as matched normal and tumor tissue, or treated/untreated cells. Elitox is an information extraction procedure which extracts RT from an original MS-MS. PAM(IS) is a procedure used to acquire internal standard information (m/z values, intensity, and retention times). PAM Wrapper is a procedure that integrates the information contained within the precursor list, Elitox, and PAM IS. RT correction uses data derived from any internal standards (PAM IS, RT and m/z values) as well as RT from original MS-MS (Elitox) to predict the retention times (RT) from MS data in an LC-MS survey and provides m/z values and retention time correction. QC checks are quality checks to assure the process is running correctly, e.g., whether it needs to be adjusted or aborted based on expected data characteristics.

FIG. 14 shows an estimation of a PAM Processing Time for a specific sample.

FIG. 15 shows a flow chart for a Peptide Hunter Module processing multiple samples (a reference sample and a treated sample in this example), comprising a Peptide Hunter Module, a Peptide Merger Module (PMM), a Band Merger Module (BMM), and a Differential Abundance Module (DAM).

FIG. 16 shows an LC-MS survey scan with expansion around 700 m/z on which PHM was run.

FIG. 17 shows the m/z values for peptides identified by PHM, as well as their charge and calculated abundance. Compare, for example, the abundance of the +1 peptide at 701.3402 to the +3 peptide at 699.9900 in the expansion of FIG. 16 to the data of this figure.

FIG. 18 shows a scatter plot of abundance data (log scale) for predicted full-length proteins (clustered peptides) comparing proteins from a normal sample to a matched tumor sample produced using PAM. The Na/K ATPase protein corresponds to a known plasma membrane (PM) marker that is not expected to change expression between normal cells and tumor cells, while CEA (carcinoembryonic antigen) is a protein known to be up-regulated in tumors.

FIG. 19 shows a scatter plot of individual peptide abundances (log scale) from a normal sample and a matched produced using PAM.

### DETAILED DESCRIPTION OF THE INVENTION

The invention features methods utilizing mass spectrometry and software to measure the abundance of a biomolecule, qualitatively or quantitatively, or both. In one application, the methods and systems of the invention are used to compare a large number of peptides present in two or more samples in order, e.g., to determine variations in relative expression levels or to identify peptides for which ratios of relative expression are above or below pre-set values. Statistical analysis of expression profiles are then used to identify peptide markers, e.g., for disease diagnostics and drug discovery.

Biological Samples

Using the methods of the invention, an expression profile of a biomolecule is monitored in a biological sample. Exemplary biomolecules useful in the methods of the invention include any organic molecule that is present in a biological sample, e.g., peptides, polypeptides, proteins, post-translationally modified peptides (e.g., glycosylated, phosphorylated, or acylated peptides), oligosaccharides and polysaccharides, lipids, nucleic acids, and metabolites. Virtually any biological sample is useful in the methods of the invention, including, without limitation, any solid or fluid sample obtained from, excreted by, or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). A biological sample may be a biological fluid obtained from any location (such as blood, plasma, serum, urine, bile, cerebrospinal fluid, aqueous or vitreous humor, or any bodily secretion), an exudate (such as fluid obtained from an abscess or any other site of infection or inflammation), or fluid obtained from a joint (such as a normal joint or a joint affected by disease such as rheumatoid arthritis). Alternatively, a biological sample can be obtained from any organ or tissue (including a biopsy or autopsy specimen) or may comprise cells (whether primary cells or cultured cells) or medium conditioned by any cell, tissue, or organ. If desired, the biological sample is subjected to preliminary processing, including preliminary separation techniques. For example, cells or tissues can be extracted and subjected to subcellular fractionation for separate analysis of biomolecules in distinct subcellular fractions, e.g., proteins or drugs found in different parts of the cell. Such exemplary fractionation methods are described in De Duve ((1965) J. Theor. Biol. 6: 33-59).

When analyzing proteins, a biological sample, if desired, is purified to reduce the amount of any non-peptidic materials present. Moreover, if desired, protein-containing samples are cleaved to produce smaller peptides for analysis. Cleavage of the peptides is generally accomplished enzymatically, e.g., by digestion with trypsin, elastase, or chymotrypsin, or chemically, e.g., by cyanogen bromide. The cleavage at specific locations in a protein allows the prediction of the masses of the smaller peptides produced if the sequences of these peptides are known. All samples that are to be compared typically are treated in the same manner.

A reference sample, if desired, is also included when performing the methods described herein. This reference sample typically includes known amounts of biomolecules or may be derived from a known source, e.g., a non-diseased tissue. The reference sample may be synthesized from known biomolecules. Additionally, unknown samples may be compared to the reference sample to determine a relative abundance. Reference samples may also be combined with other samples to act as internal standards where appropriate.

Separation of Biomolecules

A wide variety of techniques for separating any of the aforementioned biomolecules are well known to those skilled in the art (see, for example, Laemmli Nature 1970, 227:680-685; Washburn et al., Nat. Biotechnol. 2001, 19:242-7; Schaggeret al., Anal. Biochem. 1991, 199:223-31) and may be employed according to the present invention.

In one application, the methods of the invention are used to study complex mixtures of proteins. By way of example, mixtures of proteins may be separated on the basis of isoelectric point (e.g., by chromatofocusing or isoelectric focusing), of electrophoretic mobility (e.g., by non-denaturing electrophoresis or by electrophoresis in the presence of a denaturing agent such as urea or sodium dodecyl sulfate (SDS), with or without prior exposure to a reducing agent such as 2-mercaptoethanol or dithiothreitol), by chromatography, including LC, FPLC, and HPLC, on any suitable matrix (e.g., gel filtration chromatography, ion exchange chromatography, reverse phase chromatography, or affinity chromatography, for instance with an immobilized antibody or lectin or immunoglobins immobilized on magnetic beads), or by centrifugation (e.g., isopycnic centrifugation or velocity centrifugation).

In some cases, two different peptides may have the same mass within the resolution of a mass spectrometer, rendering determination of abundances for those two peptides difficult. Separating the peptides before analysis by mass spectrometry allows for the resolution of the abundances of two peptides with the same mass. Although many spectra for the fractions of the separation may then be obtained, these spectra typically have a reduced number of ion peaks from the peptides, which simplifies the analysis of a given spectrum.

In one embodiment, a mixture of proteins is separated by 1D gel electrophoresis according to methods known in the art. The lane containing the separated proteins is excised from the gel and divided into fractions. The proteins are then digested enzymatically. The peptides produced in each fraction are then analyzed by mass spectrometry. In another embodiment, peptides are separated by 2D gel electrophoresis according to methods known in the art. The proteins are then digested enzymatically, and the digested peptides produced in each fraction are then excised and analyzed by mass spectrometry. In still another embodiment peptides are separated by liquid chromatography (LC) by methods known in the art, including, but not limited to, multidimensional LC. LC fractions may be collected and analyzed or the effluent may be coupled directly into a mass spectrometer for real-time analysis. LC may also be used to separate further the fractions obtained by gel electrophoresis. Recording the retention time (RT) of a peptide in LC enables the identification of that peptide in multiple fractions. This identification is typically useful for obtaining an accurate abundance. In any of the above embodiments, a given peptide may be present in more than one fraction depending on how the fractions were obtained.

Mass Spectrometry

Exemplary methods for analyzing biomolecules using mass spectrometry techniques are well known in the art (see Godovac-Zimmermann et al. (2001) Mass Spectrom. Rev. 20: 1-57 (PMID: 10344271); Gygi et al. (2000) Proc. Natl. Acad. Sci. U.S.A. 97: 9390-9395 (PMID: 10920198)).

In applications involving peptides, the peptides are ionized, e.g., by electrospray ionization, before entering the mass spectrometer, and different types of mass spectra, if desired, are then obtained. The exact type of mass spectrometer is not critical to the methods disclosed herein. For example, in a survey scan, mass spectra of the charged peptides in a sample are recorded. Furthermore, the amino acid sequences of one or more peptides may be determined by a suitable mass spectrometry technique, such as matrix-assisted laser desorption/ionization combined with time-of-flight mass analysis (MALDI-TOF MS), electrospray ionization mass spectrometry (ESI MS), or tandem mass spectrometry (MS/MS). In a MS/MS scan, specific ions detected in the survey scan are selected to enter a collision chamber. The ability to define the ions for MS/MS allows data to be acquired for specific precursors, while potentially excluding other precursors. The ions may be defined by a predetermined list or by a query. Lists may be inclusion lists (i.e., ions on the list are subjected to MS/MS) or exclusion (i.e., ions on the list are not subjected to MS/MS). The series of fragments that is generated in the collision chamber is then analyzed again by mass spectrometry, and the resulting spectrum is recorded and may be used to identify the amino acid sequence of the particular peptide. This sequence, together with other information such as the peptide mass, may then be used, e.g., to identify a protein. The ions subjected to MS/MS cycles may be user defined or determined automatically by the spectrometer.

Mass Intensity Profiling System (MIPS)

Software to analyze mass spectra is typically used to identify the biomolecule from which an ion was derived. A mass spectrum, however, also includes information on the relative intensity of an ion as reflected by its corresponding ion intensity (e.g., ion counts per second). Moreover, a mass spectrum typically includes a large amount of information corresponding to ion intensities from several charge states or several isotopes of a biomolecule. As is described herein, an automated approach allows the processing of mass spectra recorded for one or more samples so that a comprehensive characterization of the biomolecules in that sample is achieved.

As is described in more detail below, the software used in the methods herein automatically identifies ion signals (e.g., peptide ion signals) in the spectra. A biomolecular abundance measure is then calculated by a variety of methods. For example, the intensity of a specific subset of biomolecular ions (such as all ions with +2 charge), the intensities of all ions derived from one isotope of a biomolecule (such as only the ¹²C, ¹H, ¹⁴N, and ¹⁶O-containing biomolecules), the intensities of a certain subset of isotopes of a biomolecule, the intensities of all isotopes, or any combination thereof can be integrated.

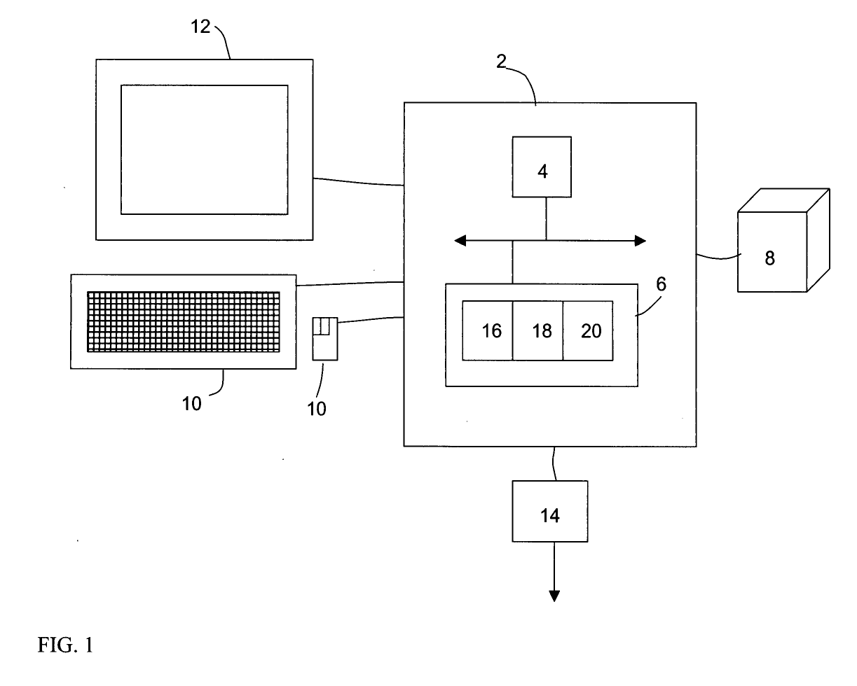

The methods described herein are implemented using virtually any computer system and according to the following exemplary programs. FIG. 1 shows an exemplary computer system. Computer system 2 includes internal and external components. The internal components include a processor 4 coupled to a memory 6. The external components include a mass-storage device 8, e.g., a hard disk drive, user input devices 10, e.g., a keyboard and a mouse, a display 12, e.g., a monitor, and usually, a network link 14 capable of connecting the computer system to other computers to allow sharing of data and processing tasks. Programs are loaded into the memory 6 of this system 2 during operation. These programs include an operating system 16, e.g., Microsoft Windows, which manages the computer system, software 18 that encodes common languages and functions to assist programs that implement the methods of this invention, and software 20 that encodes the methods of the invention in a procedural language or symbolic package. Languages that can be used to program the methods include, without limitation, Visual C/C+from Microsoft. In preferred applications, the methods of the invention are programmed in mathematical software packages that allow symbolic entry of equations and high-level specification of processing, including procedures used in the execution of the programs, thereby freeing a user of the need to program procedurally individual equations or procedures. An exemplary mathematical software package useful for this purpose is Matlab from Mathworks (Natick, Mass.). Using the Matlab software, one can also apply the Parallel Virtual Machine (PVM) module and Message-Passing Interface (MPI), which supports processing on multiple processors. This implementation of PVM and MPI with the methods herein is accomplished using methods known in the art. Alternatively, the software or a portion thereof is encoded in dedicated circuitry by methods known in the art.

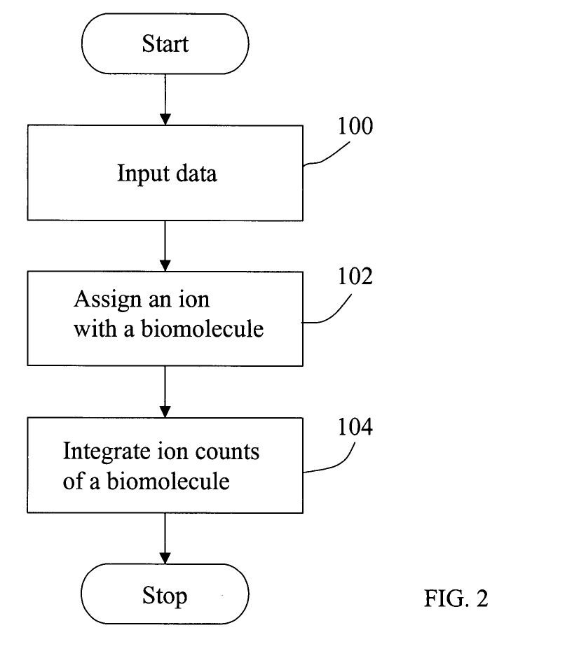

FIG. 2 shows a computer implemented flowchart of a method of determining an abundance of a biomolecule in a sample. In particular, FIG. 2 describes determining an abundance of one biomolecule in a sample. The method, however, may also be used to determine an abundance of more than one biomolecule in the sample.

At step 100, mass spectrometry measurements are input into the program. These measurements may include MS data, MS/MS data, a plurality of scans from a separation of a biomolecule or fragment thereof, and/or structural or sequence information, e.g., obtained from searching a database. At step 102, ions measured by a mass spectrometer are assigned to a biomolecule. The biomolecule may be a fragment of a larger biomolecule. The assignation can be based on determining a mass or charge state of the ion as is described herein. The assignation can alternatively be based on sequence or structural information obtained, for example, from MS/MS and database searching as described herein. At step 104, ion counts, corresponding to intensities of ions, are integrated. Ion counts for one ion peak that are spread, for example, over a range of m/z values or scans are summed to determine an integrated intensity for each peak. An integration of several peaks that are generated from the same biomolecule, for example, from charge states, isotopes, scans, and fragments of a larger biomolecule, or a subset thereof, may also occur. The ion counts may also be normalized with one or more internal standards. The integrated ion counts from a sample may be compared to ion counts from another sample, e.g., a reference sample, to determine an abundance of the biomolecule in the sample relative to another sample. The integrated ion counts may also be normalized with an absolute standard for that biomolecule to determine an absolute abundance of the biomolecule in the sample. The results of the analysis may be displayed to the user, e.g., on a monitor, or stored in memory. Further analysis of the data may then occur such as statistical analysis of the calculated abundances.

In one application, the invention features computer implemented modules for studying proteins. Such modules are described here as exemplars of the methods of the invention. Other biomolecules may be studied using similar modules. As is described below, the Peptide Abundance Module (PAM) determines abundances of known peptides (e.g., precursors for which MS/MS spectra have produced sequence information, or predicted peptides, such as the constituent peptides of a full-length protein or of a theoretical sequence, for which a mass and/or retention time can be predicted), and the Peptide Hunter Module (PHM) identifies and determines abundances of unknown peptides (e.g., precursors that have not yet been matched with MS/MS spectra, or for which MS/MS spectra have not yet been determined). The PHM and PAM modules of MIPS, if desired, are run simultaneously in a multiprocessing environment to reduce the time required for analysis. The multiprocessing environment, for example, includes a cluster of systems (e.g., Linux-based PCs) or servers with multiple processors (e.g., from Sun Microsystems), and the methods herein are implemented onto such distributed networks using methods known in the art (see Taylor et al. (1997) Journal of Parallel and Distributed Computing 45: 166-175).

The PAM and PHM offer significantly increased speed of analysis compared to performing the methods herein manually. For example, the PAM applied to finding one protein with five tryptic peptides in five samples requires approximately seven minutes to obtain an abundance of the protein. Manually, the process requires several days. In another example, the PHM for one data set requires 15 minutes to find more than 2000 proteins, but manually, the task would require more than several hundred man hours.

Peptide Abundance Module

Mass spectrometry allows the identification of a large number of peptides in a sample, for example, from MS/MS analyses. An MS/MS cycle produces peptide sequence information on a selected peptide, which may then be used to search databases comprehensively. For example, a computer is used to search available databases for a matching amino acid sequence or for a nucleotide sequence, including an expressed sequence tag (EST), whose predicted amino acid sequence matches the experimentally determined amino acid sequence. Exemplary databases useful for this purpose include, without limitation, Genbank, EMBL, NCBI, MSDB, SWISS-PROT, TrEMBL, dbEST, Human Genome Sequence database, or a user-defined database. Sequence information on compounds in the databases that contain the selected peptide may then be used to produce a list of other peptides derived from that compound using a specified cleavage technique. The mass spectra are then searched automatically for peaks corresponding to ions, e.g., from charge states or isotopes of a predicted peptide. Intensity profiles for these ions are generated through the Peptide Abundance Module (PAM). The PAM can generate intensity profiles for peptides predicted from the method of cleavage as well as those used for database searching. Peptide peaks for which MS/MS data have been acquired are matched with respect to their respective retention times.

A comprehensive list of peptide peak areas is typically generated and corrected using internal standardization, which allows the intensity of the peptides in the sample to be expressed relative to the reference peptide. Peptide peak areas for a particular peptide, for example, across bands or fractions, charge states, fragments may be combined to facilitate comparison. Peptide peak areas from multiple peptides that are components of a compound may also be combined further to facilitate comparison. For example, the average of intensities of peptides derived from one protein may be taken as a measure for the protein abundance. A comparison of peak areas enables the identification of differences in protein abundance between experimental sets. The process is entirely automated, which facilitates data analysis.

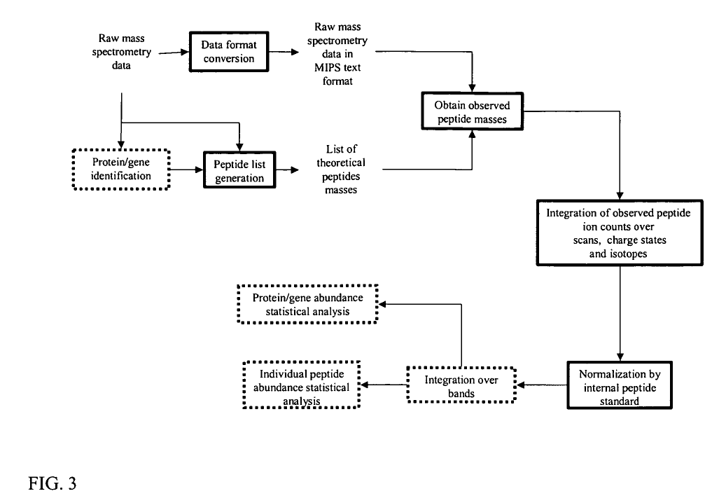

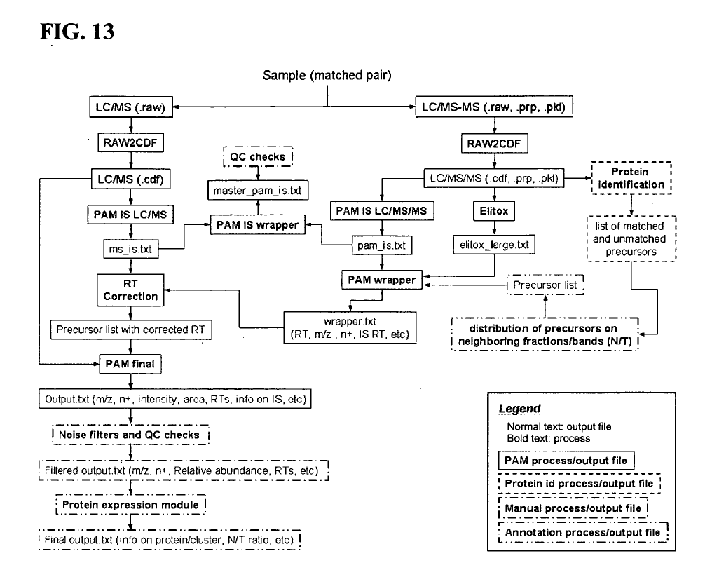

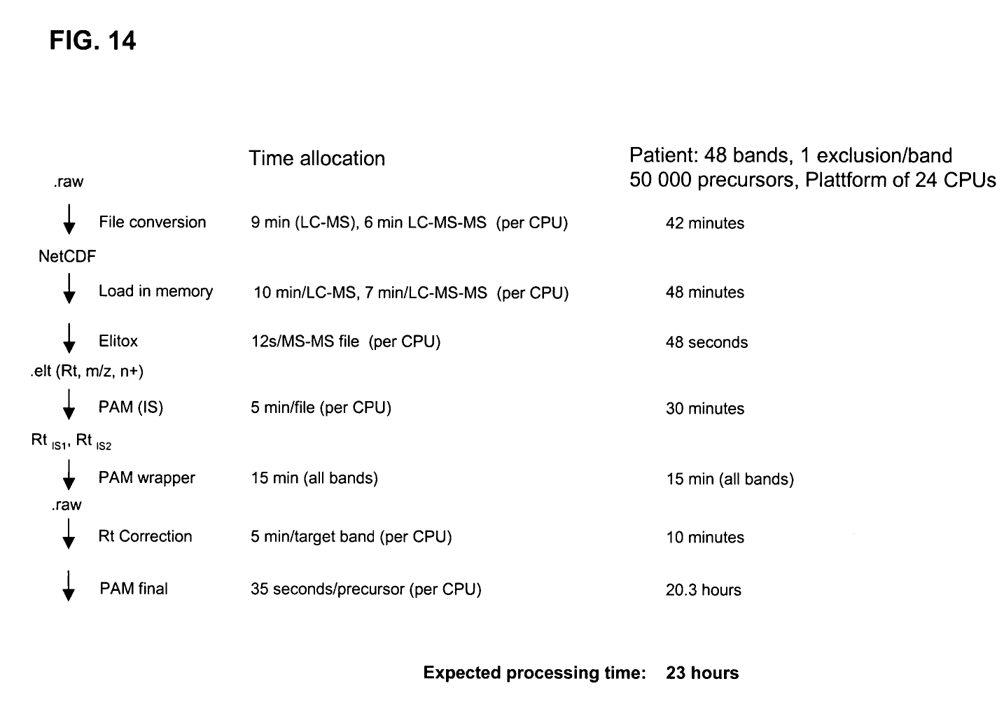

FIG. 3 shows a flowchart detailing the components of a PAM. Each component is described in detail below. A flowchart for an exemplary PAM is shown in FIG. 13. This flowchart is presented for the purpose of illustrating, not limiting, the methods of the invention. An example estimate of time for PAM processing a specific sample is illustrated in FIG. 14.

Data format conversion. The raw mass spectrometry data files typically consist of MS scans or a series of survey scans and MS/MS cycles for each fraction of the separation. Each mass spectrum corresponds, e.g., to an elution time period for LC or to a fraction for gel electrophoresis, or both. Each survey scan records the number of ions of each m/z value detected by the mass spectrometer. The raw mass spectrometry data files may be generated by various publicly available software packages including, without limitation, MassLynx from Micromass (Beverly, Mass.). To integrate MIPS with, e.g., MassLynx, software in MassLynx converts the data from the mass spectrometer, for example, into an ASCII or NetCDF format. Other software packages for obtaining mass spectrometry data have similar conversion software. Alternatively, software for data conversion is written using methods known in the art and included in the module. Optionally, data conversion, may also include merger of multiple files. File merger may also include merger of elements of the files, such as the abundances of particular precursors.

Protein/Gene Identification. The raw mass spectrometry data is submitted for compound, e.g., protein, identification using a tool such as Mascot from Matrix Science (London, United Kingdom), ProteinLynx Global Server from Micromass SEQUEST/TurboSEQUEST from Thermo Finnigan (San Jose, Calif.), or Sonar MS/MS from ProteoMetrics (New York, N.Y.). This analysis generates a list of proteins that are likely to exist in the sample under analysis.

Peptide List Generation. In this component a list of annotated theoretical peptide masses are generated from the list of identified proteins, identified genes, and the raw mass spectrometry data as follows. Cleavage (e.g., tryptic) peptides of the identified compounds may be predicted, and theoretical masses of these peptides may be generated. The mass of a peptide can then be estimated from its amino acid sequence. The mass may also be based upon the mass of a matched peptide from a sample. Other peptide masses of interest can be added to the list of theoretical peptide masses. For example, the mass of a post-translationally modified peptide (e.g., a phosphorylated or glycosylated peptide) is estimated from the unmodified mass. Adding these masses to the list allows MIPS to track the relative abundance of modified to unmodified peptides. The theoretical peptides masses may be annotated by their source (e.g., protein, gene, raw data, or modified peptide).

For normalization across samples, one or more internal peptide standards are optionally added into each sample. The theoretical peptide mass of any internal standards are then added to the theoretical peptide mass list.

Obtain Observed Peptide Masses. The peptide masses measured by the spectrometer may differ from the theoretical peptide masses depending on the accuracy of mass measurement as defined by the instrument parameters. This discrepancy can be corrected by determining the mass with the maximum ion count within a predefined range around the theoretical peptide mass according to known correction methods. This range is defined by the accuracy of the mass spectrometry instrument and also the user defined tolerance for false positives and false negatives. A list of observed peptide masses is thus generated from the list of theoretical peptide masses.

Integration of Observed Peptide Ion Counts Over Scans, Isotopes, and Charge States. To obtain the abundance of a peptide, the intensities of all ions, or a subset thereof, for that peptide are counted. In addition, the ions of a peptide that occur in different spectra because of the separation, if desired, are integrated.

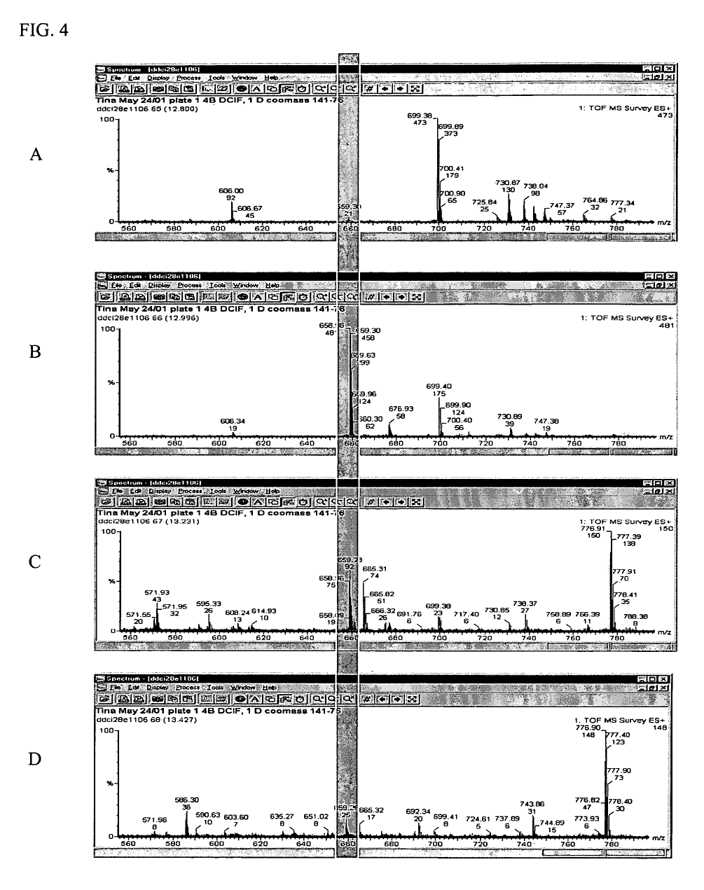

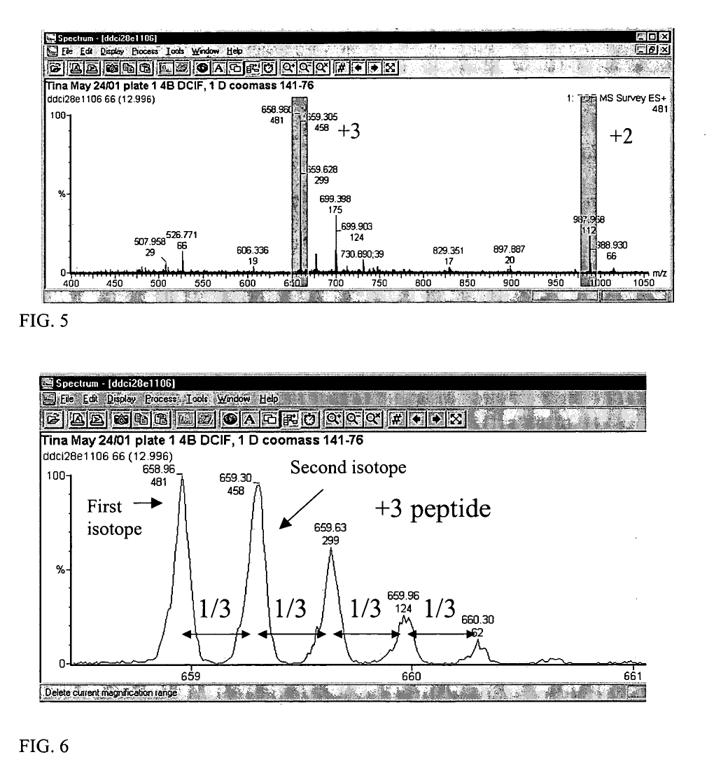

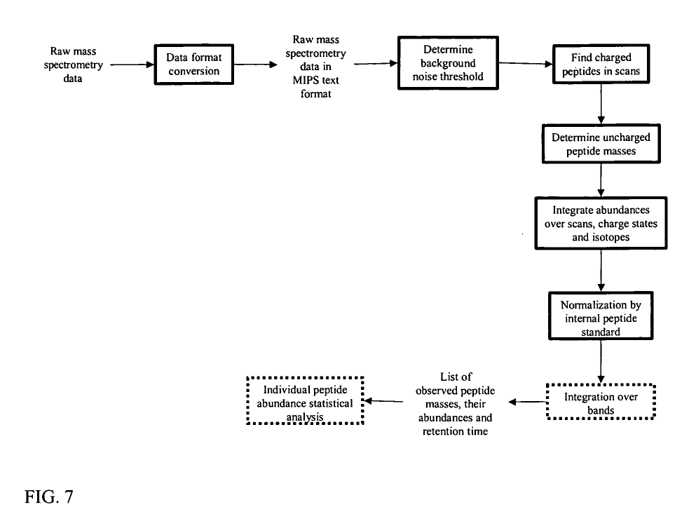

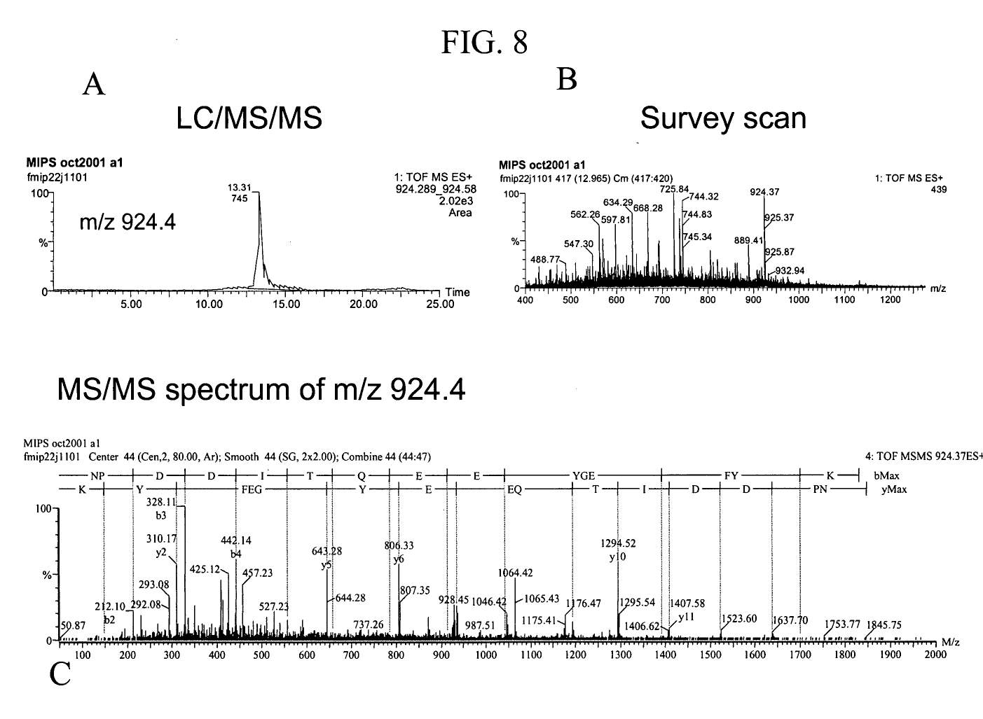

Each peptide in the sample under analysis is eluted over a series of scans within the raw mass spectrometry data (see FIG. 4). This collection of scans is referred to as the “scan window” of the peptide. Each scan in this window corresponds to a fraction from the separation, e.g., an elution time from LC. For LC, a retention time for a peptide is determined based on the intensity of an ion corresponding to that peptide as a function of scan number, which corresponds to time.

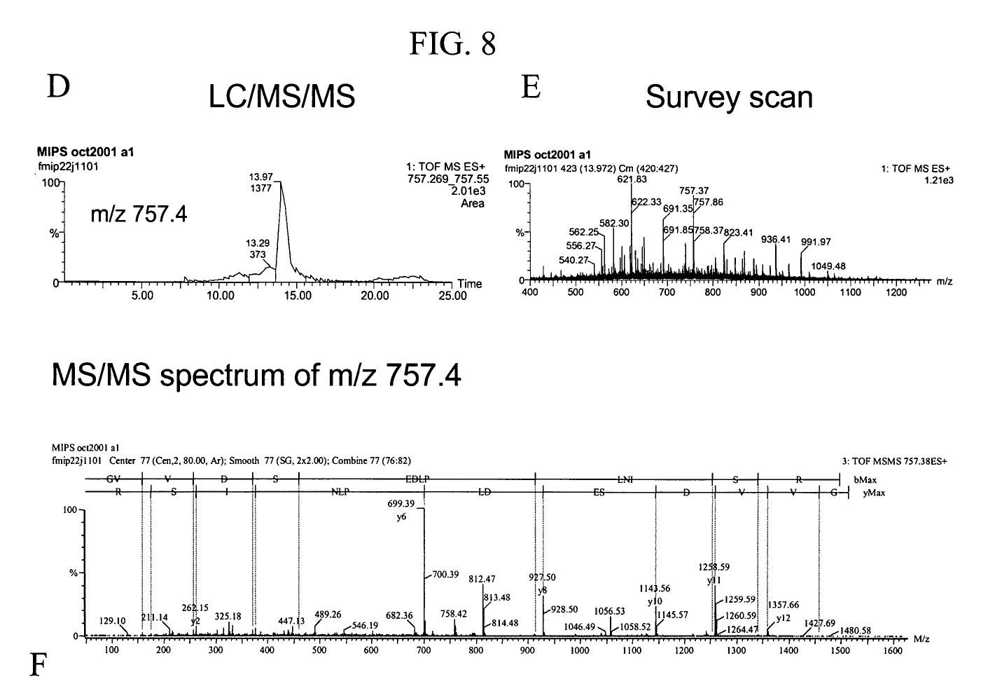

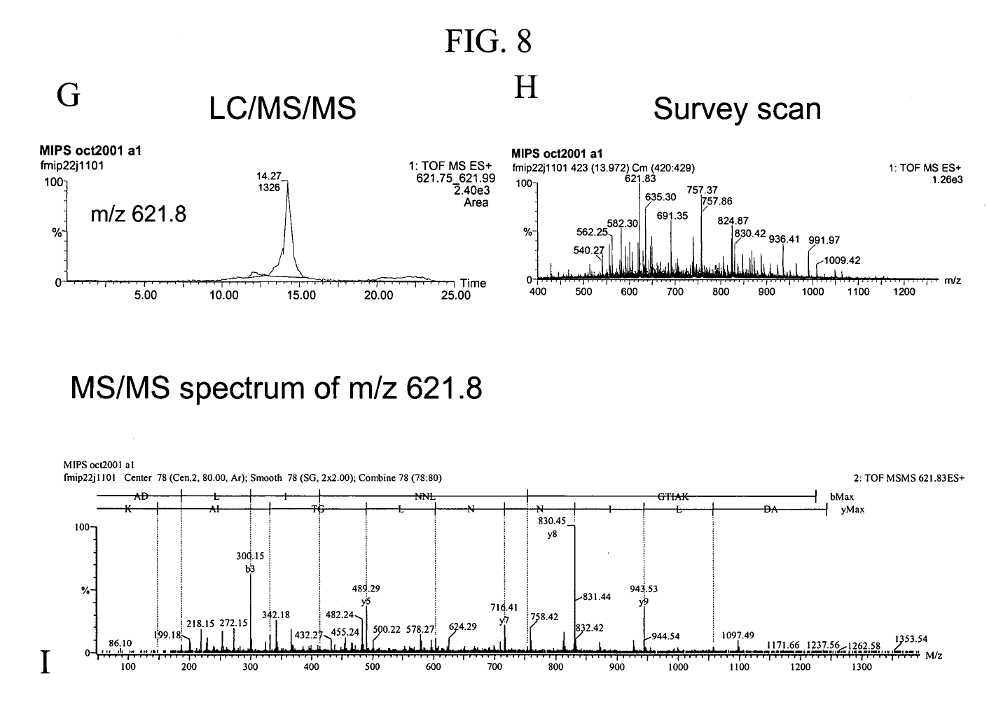

In each of the scans within a peptide's scan window, the peptide occurs in multiple charge states, typically +1, +2, +3, or +4. Scans record mass/charge (m/z). For example, the ion peak for a doubly-protonated peptide (charge +2) is (1974+(2×1.0078))/2=988.0078 when the uncharged mass is 1974 (see FIG. 5) and when the peptide is ionized by electrospray ionization. For each charge state of a peptide in a scan, the ions of the peptide are distributed over multiple isotopes. These peptide isotopes are the result of the natural abundances of isotopes of the constituent atoms. Five or more isotopes per charge state may be present. The m/z of these isotopes is predicted from the mass and charge of the peptide. For example, a peptide with mass 1974 and a charge of +3 has a m/z of 659 (see FIG. 6).

For each isotope examined, an integration window is defined, over which ion counts are summed. This window is typically required because of the resolution of the mass spectrometer and the accuracy in mass measurement. The width of this window depends, for example, on the peak width at a predefined height for an isotopic peak of the peptide. For a Gaussian peak, the ratio of peak width at half height (PW₅₀) and peak width at 5% height (PW₅) should be constant and equal to approximately 2.2. The relationship linking PW₅ to the m/z value is represented by PW₅=(2.2×m/z)/RES where RES is the instrumental resolution as defined at PW₅₀. Once the PW₅ is calculated for a peak, ion counts from m/z−PW₅/2 to M/Z+PW₅/2 are summed, for example, by adding the measured intensities for data points falling within the window, to produce the integrated intensity of that peak.

The procedure for determining the abundance of a peptide with observed mass is to sum the ion counts over isotopes of all charge states, or a subset thereof, in scans where the sum of the ion counts are above a predefined signal to noise threshold.

The retention time of each peptide is also obtained in this process. If two peptides of the same mass but significantly different retention times are detected, then two separate entries for these peptides are created since they are likely different peptides that happen to have the same mass. This duplicity may be noted in the data.

Normalization. The abundances of the observed peptide masses may be corrected for instrument variation using the abundance of one or more internal standards, e.g., Leu-enkephalin. A standard may be added from an external source (e.g., a synthetic peptide standard) or may be intrinsic to the sample (e.g., peptides derived from a protein marker believed to remain constant across conditions to be compared). Preferably, several such standards are used across the range of retention times. An absolute abundance of a peptide may be determined using intensity data from purified or synthetic versions of that peptide. Optionally, a sub-component of the module can be used to acquire data about internal standards, including, but not limited to, m/z values, intensity, and retention times. Such data may be used in combination with known reference values to correct and/or predict the corresponding values for the standards themselves, as well as of other peptides and precursors. For example, data derived from internal standards (such as, RT and m/z values) as well RT from original MS-MS peptide data can be used to predict the retention times (RT) of peptides in MS data from the LC-MS survey. Data about internal standards and peptides with m/z values and corrected retention times may be produced for a subset or a in file-wide data integration 1 correction.

Integration Over Fractions or Bands. If samples analyzed by mass spectrometry are excised from 1D gels, the abundance of an observed peptide is usually integrated over neighboring bands since the peptide can appear in several bands. The same peptide in neighboring bands is then identified, e.g., by mass, retention time, and MS/MS. If samples are analyzed by multidimensional LC (e.g., 2D), the abundance is typically integrated over salt fractions.

Protein/Gene Abundance Statistical Analyses. The abundance of a protein or gene is proportional to the abundance of its peptide abundances. Statistical analyses may be performed to determine the relative abundance of a protein or gene across samples. For example, the distribution of peptide abundances in normal tissue is compared to the distribution of the peptide abundances in diseased tissue using a statistical test of significance. In addition, the relative abundance of post-translationally modified and unmodified proteins within a sample may be determined from the relative abundance of modified to unmodified peptides. Abundances of isoforms of a protein may also be determined. Similarly, the relative abundance of splice variants of a gene may be determined.

Individual Peptide Abundance Statistical Analyses. Observed peptides not assigned to a protein or gene through protein identification may also be compared across samples. Significant differences in abundance across samples indicate interesting peptides for further analysis.

Those peptides that occur in the raw mass spectrometry data but not within any of the identified proteins or genes may be obtained, e.g., from the raw mass spectrometry data, e.g., using pattern detection methods.

Peptide Hunter Module

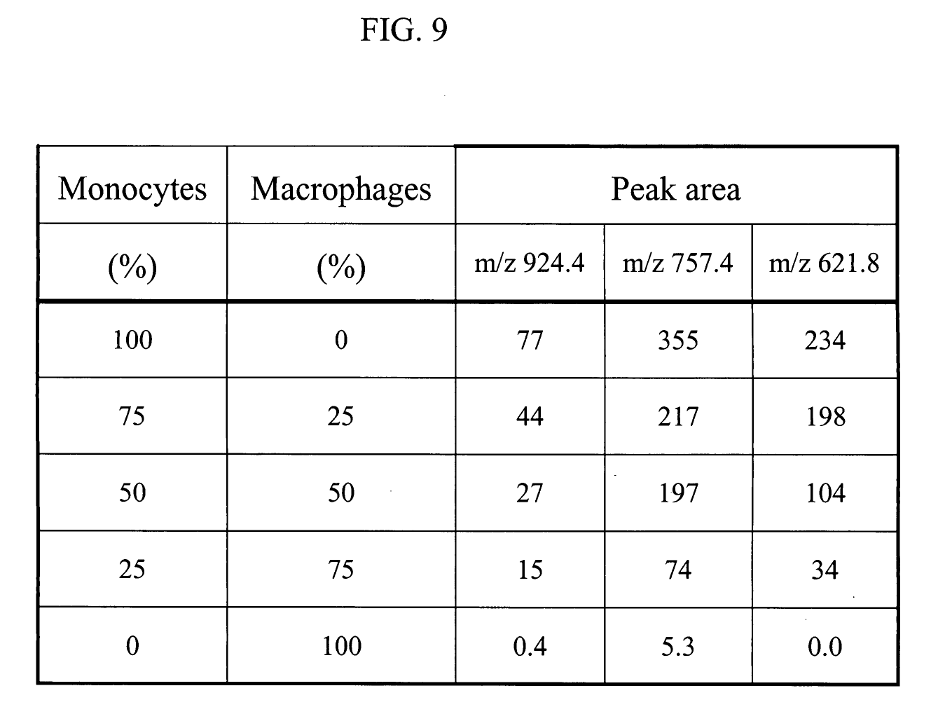

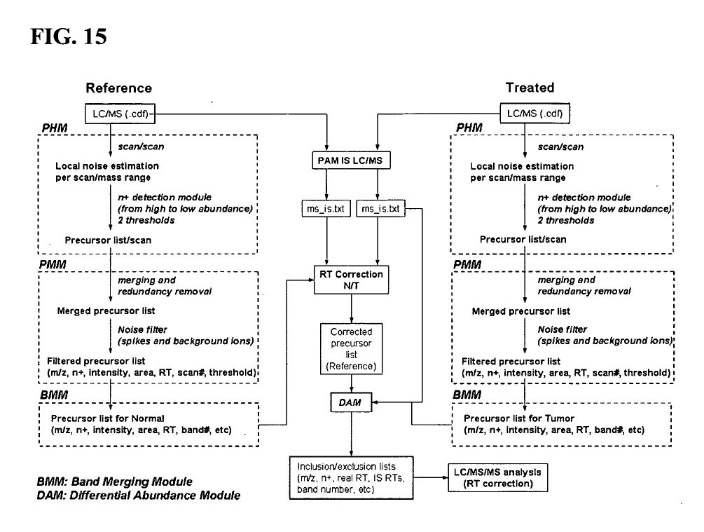

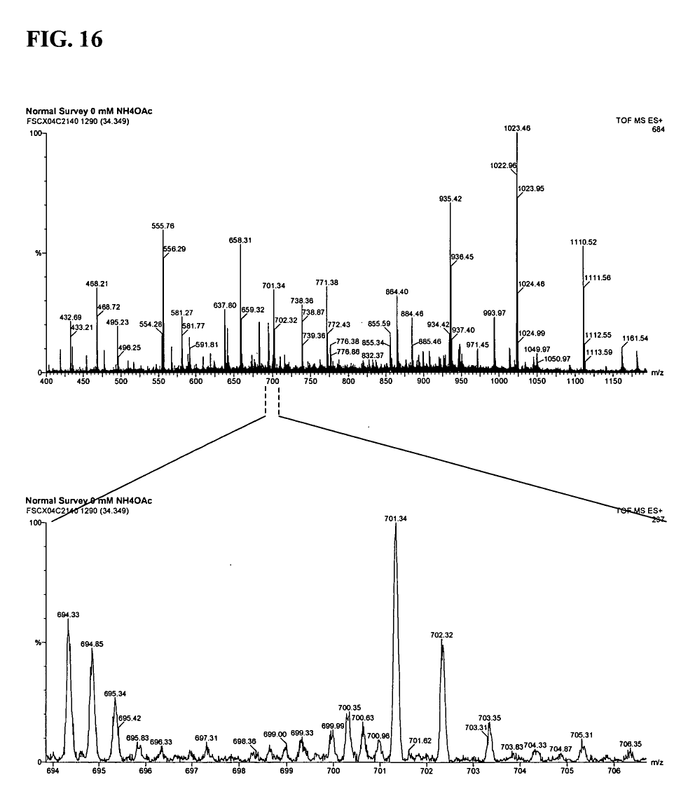

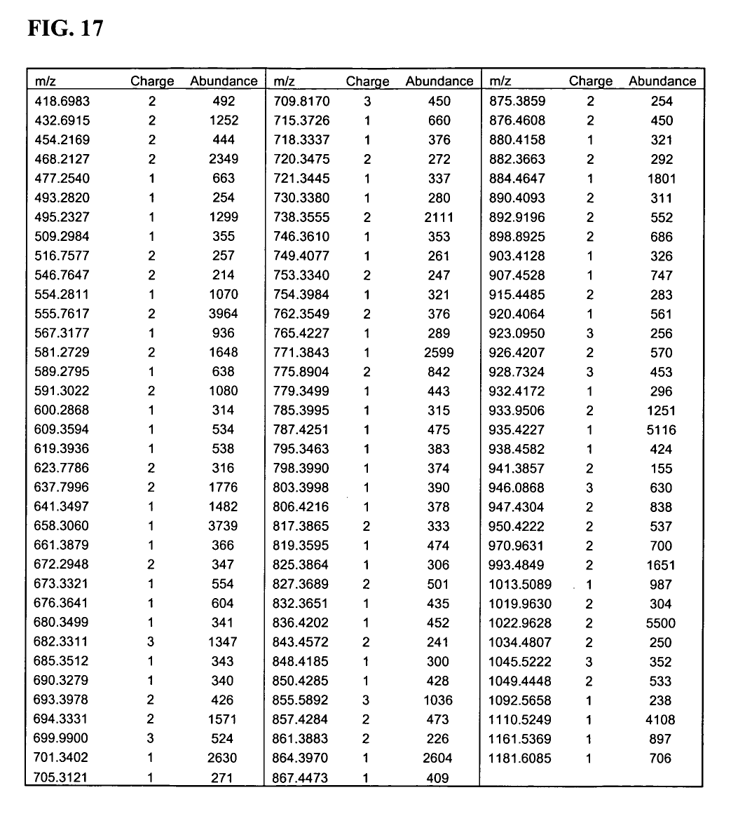

The Peptide Hunter Module (PHM) differs from the Peptide Abundance Module in that proteins t and their corresponding tryptic peptides are “precursors” and need not have been identified, such as by being sequenced by MS-MS, prior to using PHM. The PHM therefore mines raw mass spectrometry data for peptides, calculates their abundance (see FIGS. 16 and 17 for an example), and may render them comparable between samples in part by correction of their mass, intensity, and retention times through the use of internal standards. PHM allows the identification of full-length proteins a peptide may be from through peptide mass fingerprinting. PHM also permits the generation of a list of precursors for further identification by a round of MS/MS. FIG. 7 is a flowchart detailing the components of a Peptide Hunter Module (PHM). Solid rectangles represent processing components of MIPS, dashed rectangles represent processing components that are not within MIPS and entries without a rectangle are data files. A flowchart for an exemplary PHM is shown in FIG. 15. Each component is described in detail below. This flowchart is presented for the purpose of illustrating, not limiting, the methods of the invention.

Data Format Conversion. Data format conversion occurs as is described above in the PAM.

Determination of a Threshold. Since the PHM mines the survey scans in the raw mass spectrometry data for evidence of peptides, a threshold of ion intensity is defined to differentiate signal from peptide ions from those of noise. This threshold is estimated for all scans by using methods known in the arts, such methods include, without limitation, the method of Maximum Entropy.

Find Charge States of Peptides in Survey Scans. A survey scan of raw mass spectrometry data is searched for evidence of charged states of peptides. The pattern of a charge state of a peptide is depicted in FIG. 6. Each charge state consists of a pattern of isotopic peaks. The isotopes of the charged state are separated in a spectrum by 1.0034/z, where z is the charge of the peptide. The “first isotope” of a charge state can be located at a specific m/z value with an isotope located at ((m/z value)+1.0034/z), but without an isotope located at ((m/z value)−1.0034/z) in the spectrum. The second isotope can be located at ((m/z value)+1.0034/z) in the spectrum, and so on.

To identify a charge state for a peptide, a data point corresponding to an m/z can be selected, e.g., on the basis of intensity, from the data in a spectrum. The data can then be searched systematically for neighboring peaks separated by 1.0034/z for a defined number of charges, e.g., +4, +3, +2, and +1. The program searches an appropriate region around 1/z to compensate for uncertainty in the experimental data. The charges can be searched in order from highest to lowest until a peak is found. This order is typically required since, for example, a +4 charged peptide could be mistakenly interpreted as a +1 charged peptide since the +4 charged peptide and the +1 charged peptide both have isotopes at (m/z value of first isotope +1). If no neighboring peaks are found, a charge state cannot be assigned using this method. If a neighboring peak is present, for example, at m/z+0.33, then the charge state can be identified by the separation, which in this case corresponds to the +3 state (FIG. 6). Isotopes in a charge state are identified based on one peak and the separation (1.0034/z). Isotopes of a charge state may be assigned to the same mass or m/z, e.g., the mass or m/z of the first isotope, to facilitate integration of peaks originating from the same peptide. The search may require that a peak be a first isotope, and that the second isotope be at least a specified fraction (possibly greater than 1) of the first isotope. Once a charge state is identified, a mass of the peptide may be calculated and used to search for other charge states from the same peptide. By using this procedure, many peaks may be identified from the initial identification of one peak.

In one embodiment, for each peak, m, in the scan, beginning with the most intense peak and progressing to the least intense peak with intensities above the threshold, t, the following steps occur. Alternatively, only a selected number are analyzed as follows. Ion counts within a window, w, around data point m are integrated to obtain abundance, A1. Ion counts within a window, w, around m+0.25 are then integrated to obtain abundance, A2. Ion counts within a window, w, around m−0.25 are then integrated to obtain abundance, A0. If A2 is greater than p×A1 and A1 is greater than q×A0, then m is the first isotope of the +4 charge state of a peptide. Otherwise, repeat the above steps with 0.25 replaced with 0.33, 0.5, and 1 to test for the +3, +2, and +1 charge states. The parameters w, t, p, and q are user defined. The threshold ensures that only peaks of sufficient intensity are examined. The parameters p and q can ensure that peak m is a first isotope by requiring that the second isotope be at least a defined fraction of the first isotope, and that another isotope is not present at ((m/z value)−1/z). Redundancy in the form of multiply identified peptides may be eliminated.

Determine Uncharged Peptide Masses. A peptide can occur in many charge states in the scans of the raw mass spectrometry data, and all or a portion of these charge states may be collected for the peptide. Charged peptides in a scan are assigned to an uncharged peptide mass using the formula P=(m/z×z)−(1.0078×z), where P is the uncharged mass, m/z is measured by the spectrometer, and z is the charge for electrospray ionization. Other ionization schemes are known in art, and the formula is modified accordingly. Software used in the PHM may also require that peptides assigned to an uncharged peptide mass have similar retention times. In the example of FIG. 6, the PHM would detect a +3 charged peptide with an uncharged mass, P=(658.96×3)−(1.0078×3)=1973.86.

Integrate Abundances Over Scans, Charge States, and Isotopes. Integrated ion counts for individual peaks may be calculated as is described in the PAM above. For each of the uncharged peptide masses, a measure of the abundance of the uncharged peptide mass may be obtained by an integration of ion counts, e.g., over scans, charge states, and isotopes, or combinations thereof. The abundance need not take into account the abundance of every ion count for a particular peptide, though a consistent subset of the possibilities, e.g., just the +2 and +3 charge states, should be used throughout the sample. For each uncharged peptide, the previous step predetermines those charge states and isotopes that correspond to the same uncharged mass. The retention time of the uncharged peptide mass can be predicted from the scan window as in the PAM described above.

Normalization by One or More Internal Standards. The abundances of the observed peptide masses are optionally corrected for instrument variation using the abundance of one or more internal standards according to standard methods, and may be performed similarly as for PAM above. If the PHM is executed after the PAM, then a second normalization is unnecessary, although it is desirably for it to be performed for ions for which no MS-MS was obtained.

Integration Over Fractions or Bands. If samples analyzed by mass spectrometry are excised from 1D gels, the abundance of an observed peptide is typically integrated over neighboring bands since the peptide might appear in several bands. The same peptide in neighboring bands is identified, e.g., by mass, retention time, and MS/MS. If samples are analyzed by multidimensional LC (e.g., 2D), the abundance is typically integrated over salt fractions.

Individual Peptide Abundance Statistical Analyses. The list of peptides masses, their abundances, and retention times are used for various analyses, such as protein identification by mass fingerprinting; protein identification, through defining peptides for a further round of MS/MS; protein identification that combines matching MS/MS and mass fingerprinting, which can increase the peptide coverage of a protein and assist in differentiating between similar proteins in a family or between splice variants and between polymorphisms; and determining low abundance peptides present in the raw mass spectrometry data, which may correspond to low abundance proteins in the sample being analyzed.

Expression Profiling

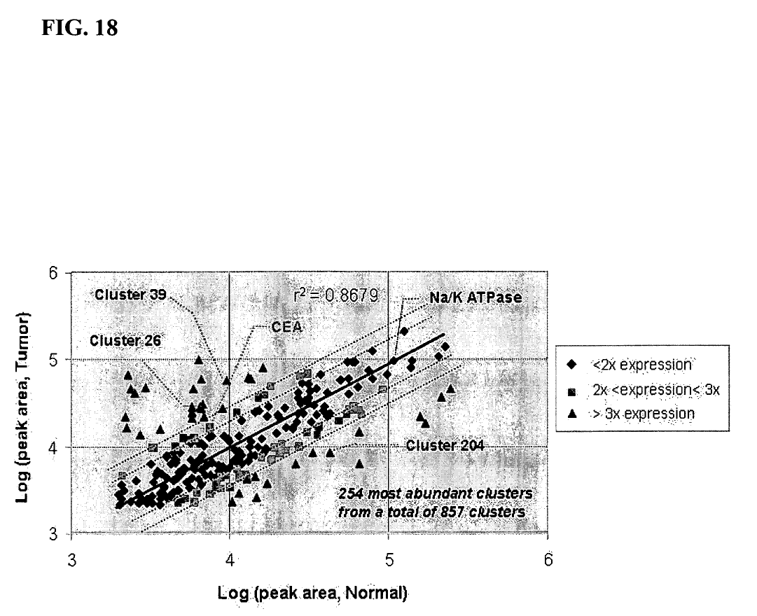

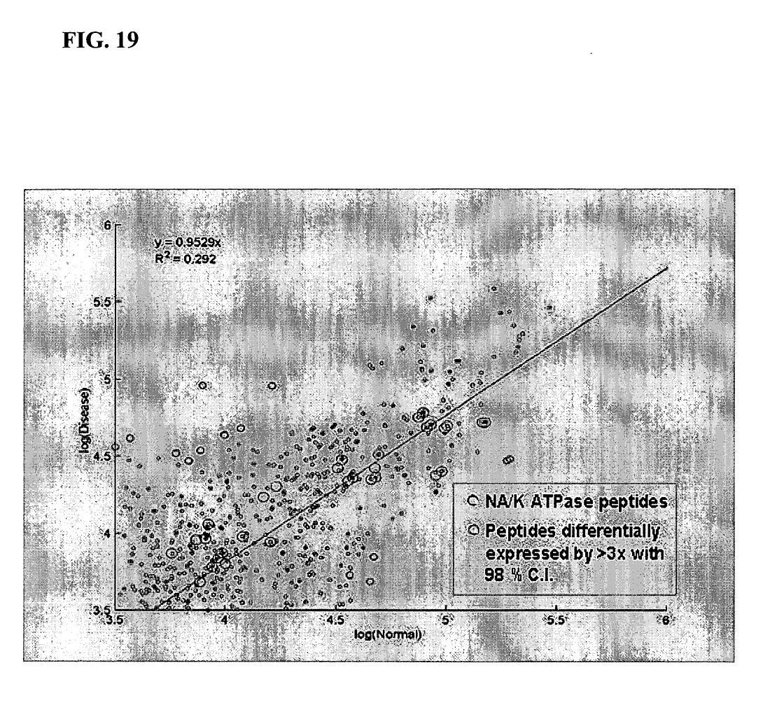

The methods of the present invention can be used to determine the relative abundance of a biomolecule or fragment thereof, e.g., proteins, in samples (see FIGS. 18 and 19). Samples being analyzed are compared to a reference sample, or samples. This comparison, or expression profile, is used, e.g., to determine if biomolecules, e.g., proteins, are present in abnormally high or low amounts compared to the reference. The determination of a difference in expression of a species in a sample relative to a reference sample is used, e.g., to diagnose disease in a patient, to determine natural variance in a population, or to determine the genotype of an individual. A comparison of protein abundances between normal and tumor cells for an individual (see FIG. 18), or across a population of patients, would be exemplary applications.

Drug Targets

Once a protein is identified in a public or private database, the gene encoding the protein is cloned and introduced into bacterial, yeast, or mammalian host cells. Where such a gene is not identified in a database, the gene encoding the protein is cloned, using a degenerate set of probes that encode an amino acid sequence of the protein as determined by the methods discussed above. Where a database contains one or more partial nucleotide sequences that encode an experimentally determined amino acid sequence of the protein, such partial nucleotide sequences (or their complement) serve as probes for cloning the gene, obviating the need to use degenerate sets.

Cells genetically engineered to express such a recombinant protein can be used in a screening program to identify other proteins or drugs that specifically interact with the recombinant protein, or to produce large quantities of the recombinant protein, e.g. for therapeutic administration.

In addition, a protein identified according to the present invention can be used to generate antibodies, for example, by administering the protein to an animal, such as a mouse, rat, or rabbit, for production of polyclonal or monoclonal antibodies using standard methods known in the art. Such antibodies are useful in diagnostic and prognostic tests and for purification of large quantities of the protein, for example, by antibody affinity chromatography. Antibodies may also be used for immunotherapy, such as might be used in the treatment of cancer.

### EXAMPLES

These aforementioned methods, and the reagents and techniques for carrying out these steps, are now described in detail using particular examples.

The examples are provided for the purpose of illustrating the invention and should not be construed as limiting.

### Example 1

### Reproducibility

A total cellular lysate of U937 cells was prepared. Fifty μg of this lysate was mixed with 30 ng of bovine serum albumin (BSA). Five samples of this mixture were prepared, and each sample was separated by SDS gel electrophoresis. A sixth sample was prepared without the addition of BSA. After electrophoresis, the gel was stained, and the band containing the BSA was excised from the gel.

Tryptic digestion of the BSA band was performed according to standard methods. Three hundred and fifty fmol of Leu-enkephalin was added to each tryptic digest as an internal standard. The peptide mixture was separated using a Waters CapLC HPLC system that was coupled to a Micromass quadropole-time of flight (Q-ToF) mass spectrometer. The conditions for the separation of this mixture were as follows: a reversed phase column (1D, 10 cm×75 μm, C 18), a flow rate of 300 nL/min, and a linear gradient of 10% to 80% acetonitrile/deionized water (containing 0.2% formic acid) in 25 minutes. The settings of the mass spectrometer were as follows: MS scan using a mass range from m/z 400 to 1500, a solvent delay of 5 minutes, a scan time of 19 minutes, a scan rate of 1 second, and an interscan delay of 0.1 second.

A theoretical list of BSA-derived peptides was generated from the known protein sequence of BSA and the known cleavage preference of trypsin. A subset of these peptides, which were detected in the mass spectra, was chosen. This list, together with the mass spectrometer data files, was entered into the PAM to generate a list of peptide abundances. The average peptide abundance was calculated as a measure of protein abundance. The average and standard deviation from the 5 replicates were calculated. For all five samples, the measured BSA abundance was close to the average, with a relative standard deviation of approximately 2 to 3%. Table 1 shows the relative abundance of BSA, human dihydropyrimidinase-related protein 2 (DHP), heat shock protein 89 (HSP89), and inosine monophosphate dehydrogenase (IMP) as determined by the PAM for five samples. DHP, HSP89, and IMP co-migrated with BSA. This experiment established that the automated analysis yields highly reproducible results when the same sample is analyzed several times.

TABLE 1

| Sample | BSA | DHP | HSP89 | IMP |
|---|---|---|---|---|
| Number | RSD 2.5% | RSD 7.6% | RSD 2.8% | 11.6% |
| 1 | 451 | 1860 | 1011 | 1795 |
| 2 | 466 | 1596 | 977 | 1769 |
| 3 | 444 | 1583 | 1028 | 2125 |
| 4 | 456 | 1622 | 975 | 1923 |
| 5 | 474 | 1801 | 1030 | 2308 |

### Example 2

### Linearity

U937 cells were cultured for 48 hours with or without 25 nM phorbol myristate acetate (PMA) to generate macrophages and untransformed cells (monocytes) using standard culturing techniques. Approximately 100 million cells suspended in 10 mM Tris/200 mM sucrose, pH 7.5 homogenization buffer were placed into a cavitations chamber, pressurized with N₂ at 1000 psi and kept on ice for 60 minutes. After each incubation period, samples were released to atmospheric pressure rapidly, and centrifuged at 900 g for 15 minutes at 4° C. (low speed centrifugation). The supernatant (post nuclear supernatant, PNS) was collected and either kept or immediately centrifuged at 40000 rpm for 60 minutes at 4° C. to produce the post nuclear membrane (PNM) samples.

Five samples of 50 μg of monocyte or macrophage proteins were prepared. The first sample contained only monocyte proteins, the second sample contained 75% monocyte and 25% macrophage proteins, the third sample contained 50% of both monocyte and macrophage protein, the fourth sample contained 25% monocyte and 75% macrophage protein, and the fifth sample contained only macrophage proteins. To each sample, 50 ng of BSA was added. These samples were separated by electrophoresis, and the gel was stained using standard methods.

From each lane, 10 bands were excised (with BSA being located approximately in the middle of the lane). Each band was analyzed by mass spectrometry as described in the previous Example. The tryptic peptide mixture was separated using a Waters CapLC HPLC system coupled to a Micromass Q-TOF mass spectrometer. The conditions for the separation of this mixture were as follows: a reversed phase column (1D, 10 cm×75 μm, C18), a flow rate of 300 nL/min and a linear gradient of 5% to 60% acetonitrile/deionized water (containing 0.2% formic acid) in 15 minutes. The mass spectrometer settings were as follows: MS and MS/MS scans using a mass range from m/z 400 to 1600, a scan time of 24 minutes, a scan rate of 1 second, and an interscan delay of 0.1 second.

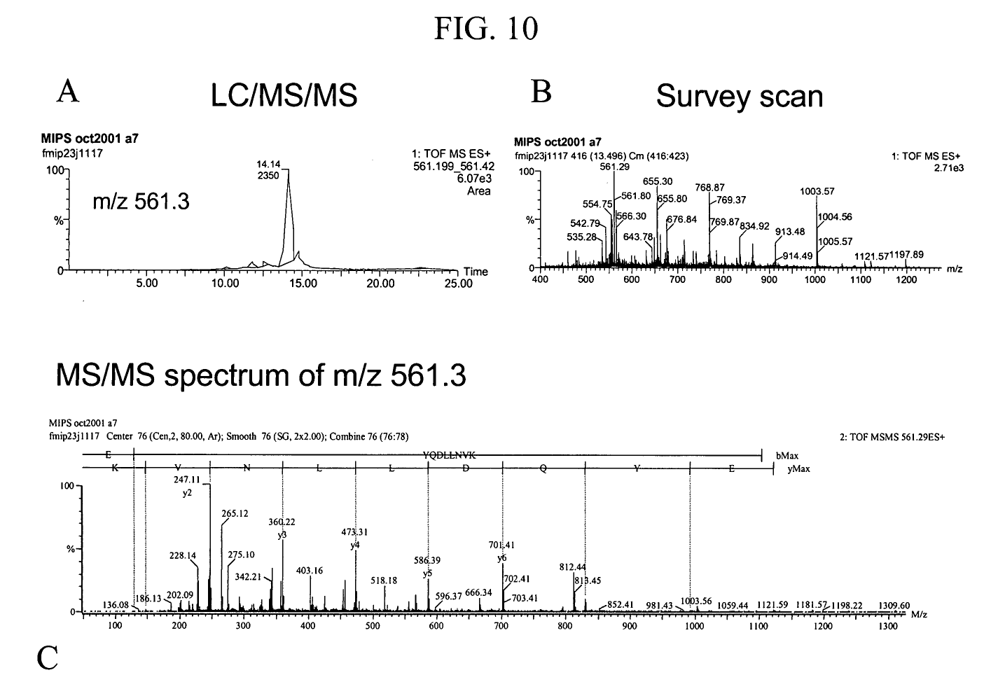

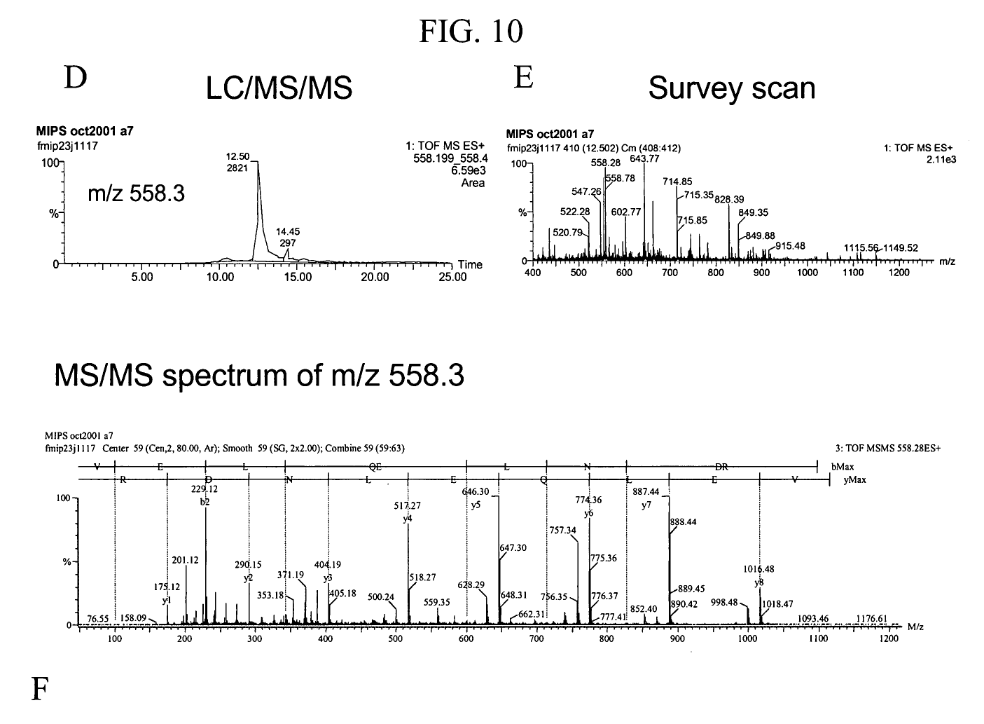

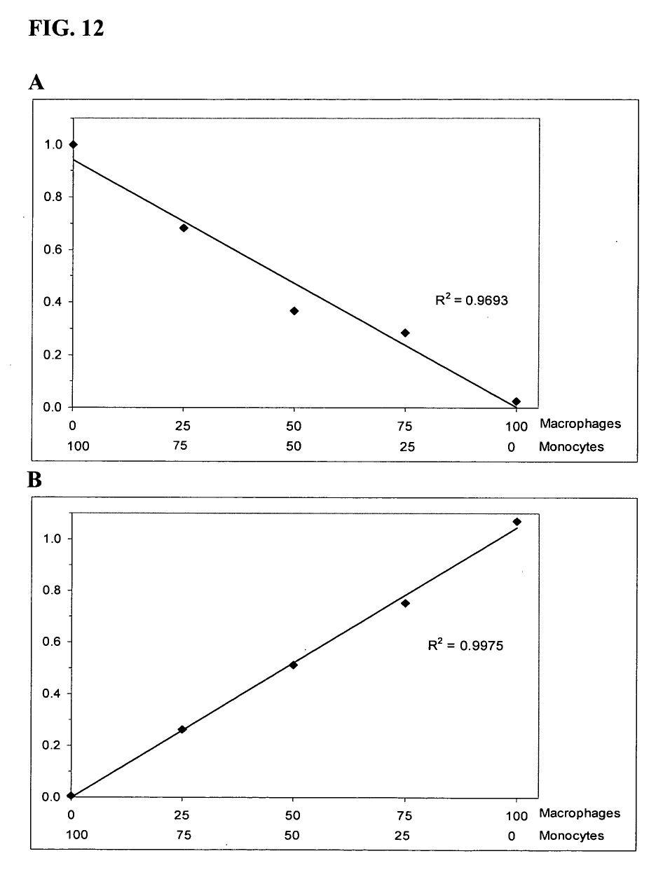

FIG. 8 shows mass spectrometry data obtained for the HSP90 protein. Three peptides were identified and characterized by their LC retention times, a MS survey scan, and MS/MS. FIG. 9 shows a table of the relative abundance of each of the peptides as a function of the concentration of macrophage proteins and monocyte proteins. The HSP90 is expressed to a greater extent in monocytes than in macrophage. FIG. 12A shows the linearity of the expression of HSP90.

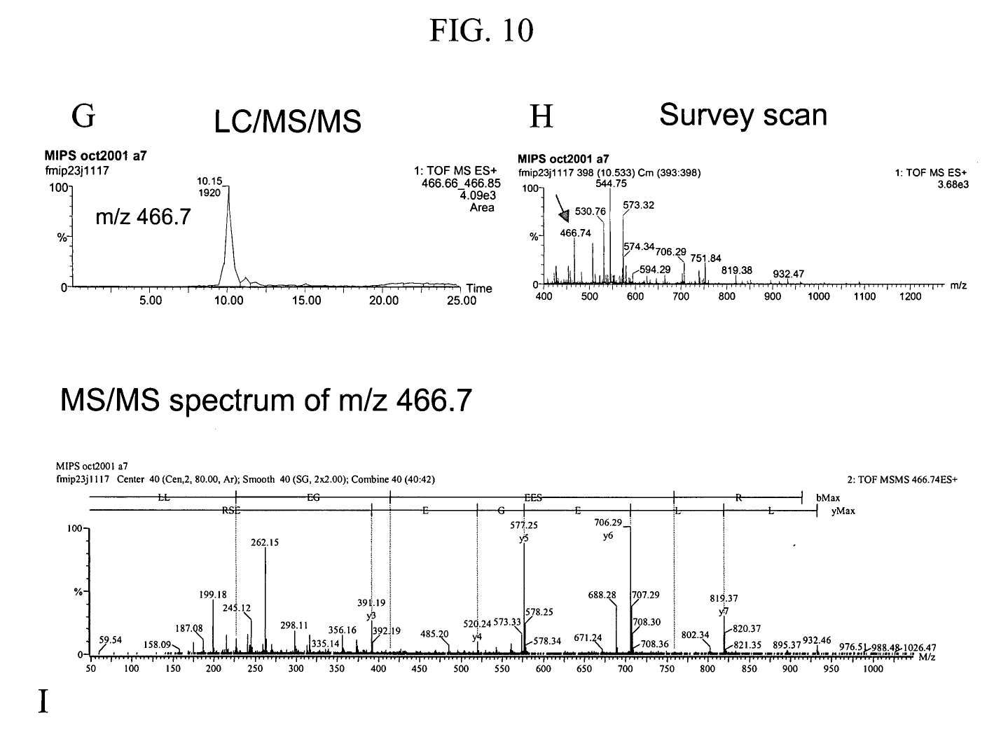

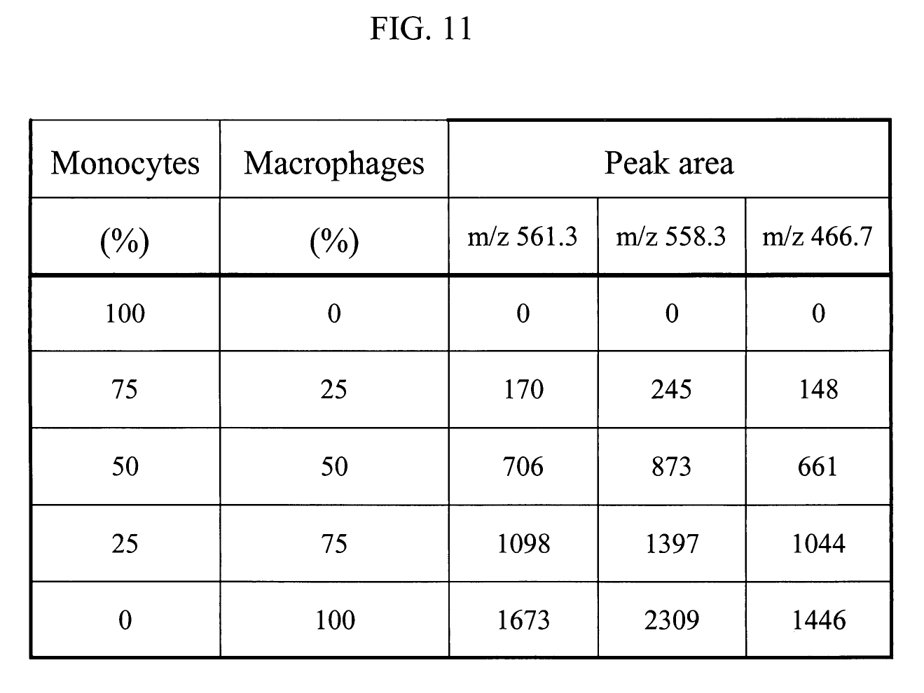

FIG. 10 shows mass spectrometry data obtained for the mutant desmin protein. Three peptides were identified and characterized by their LC retention times, a MS survey scan, and MS/MS. FIG. 11 shows a table of the relative abundance of each of the peptides as a function of the concentration of macrophage proteins and monocyte proteins. The mutant desmin is expressed to a greater extent in macrophage than in monocytes. FIG. 12B shows the linearity of the expression of mutant desmin.

### Other Embodiments

All patents, patent applications, and publications referenced herein are hereby incorporated by reference.

Other embodiments are within the following claims.

What is claimed is:

1-142. (canceled)

143. A method for displaying information on abundance of a biomolecule in a biological sample to a user, said method comprising the steps of:
a) inputting mass spectrometry data comprising ion counts for a plurality of biomolecules into a computer;
b) assigning an ion to a biomolecule;
c) integrating the ion counts of the biomolecule, thereby determining the abundance of the biomolecule in the biological sample; and
d) displaying the abundance of the biomolecule.

144. The method of claim 143, wherein step (d) further comprises storing the abundance of the biomolecule in a memory.

145. The method of claim 143, wherein said biomolecule is a protein.

146. The method of claim 145, wherein said protein is from an isolated organelle.

147. The method of claim 146, wherein said organelle is selected from the group consisting of mitochondria, chloroplasts, ER, Golgi, endosomes, lysosomes, phagosomes, peroxisomes, secretory vesicles, transport vesicles, nuclei, and plasma membrane.

148. The method of claim 145, wherein the protein is a cytosolic or cytoskeletal protein.

149. The method of claim 143, wherein said biomolecule is unlabeled.

150. The method of claim 143, wherein said biomolecule is underivatized.

151. The method of claim 142, wherein said biomolecule is a cleaved biomolecule.

152. The method of claim 151, wherein said cleaved biomolecule is unlabeled.

153. The method of claim 151, wherein said cleaved biomolecule is underivatized.

154. The method of claim 151, wherein said biomolecule is cleaved with an enzyme.

155. The method of claim 154, wherein said enzyme is trypsin.

156. The method of claim 143, wherein the plurality of biomolecules is separated prior to the acquisition of mass spectrometry data.

157. The method of claim 156, wherein separation is carried out by chromatography, electrophoresis, immunoisolation, or centrifugation.

158. The method of claim 156, wherein said biological sample includes one or more internal standards and wherein the retention time of an ion is corrected using said one or more internal standards.

159. The method of claim 143, further comprising assaying a second biological sample.

160. The method of claim 143, wherein said biological sample includes one or more internal standards.

161. The method of claim 143, where the mass spectrometry data further comprises structural or sequence information of an ion of the biomolecule.

162. The method of claim 161, wherein said structural or sequence information is obtained from MS/MS.

163. The method of claim 162, wherein a list of one or more biomolecules is provided to select an ion of a biomolecule for MS/MS analysis.

164. The method, of claim 163, wherein said list is an inclusion list.

165. The method of claim 163, wherein said list is an exclusion list.

166. The method of claim 161, wherein the structural or sequence information is used to identify a biomolecule from a database.

167. The method of claim 166, wherein the biomolecule is identified using a computer procedure selected from the group consisting of Mascot®, Protein Lynx Global Server, SEQUEST®/TurboSEQUEST, PepSEQ, SpectrumMill, or Sonar MS/MS.

168. The method of claim 166, wherein said database is the Genbank®, EMBL, NCBI, MSDB, SWISS-PROT®, TrEMBL, dbEST, or Human Genome Sequence database.

169. The method of claim 166, wherein in step (b) the ion is assigned to the biomolecule identified from said database.

170. The method of claim 143, wherein in step (b) the ion is assigned to the biomolecule by calculating an uncharged mass for the ion.

171. The method of claim 143, wherein in step (b) the ion is assigned to the biomolecule by peptide mass fingerprinting.

172. The method of claim 143, wherein in step (c) the integration is over one or more charge states, isotopes, scans, fragments of the biomolecule, fractions of a separation, or a combination thereof.

173. The method of claim 143, wherein said method further comprises calculating an abundance of the biomolecule relative to a control biological sample.

174. The method of claim 143, wherein said method further comprises calculating abundances of a plurality of biomolecules relative to a control biological sample.

175. The method of claim 173, wherein the abundance is used to diagnose a disease or condition.

176. The method of claim 173, wherein abundance is used to determine a biomolecule to target with a drug.

177. The method of claim 173, wherein an increase or decrease in abundance or the presence or absence of a biomolecule in the biological sample relative to the control biological sample is indicative of a disease or condition.

178. The method of claim 173, wherein the abundance is used to determine an amount of an isoform of a biomolecule.
